(19) United States
(12) Patent Application Publication
(10) Pub. No.: US 2018/0258424 A1
(43) Pub. Date: Sep. 13, 2018

(54) CRISPR COMPOSITIONS AND METHODS OF USING THE SAME FOR GENE THERAPY

(71) Applicant: CODA BIOTHERAPEUTICS, INC., South San Francisco, CA (US)

(72) Inventors: Kenneth P. GREENBERG, Berkeley, CA (US); Mitchell H. FINER, Stow, MA (US)

(21) Appl. No.: 15/977,192

(22) Filed: May 11, 2018

Related U.S. Application Data
(63) Continuation of application No. PCT/US2016/061633, filed on Nov. 11, 2016.
(60) Provisional application No. 62/254,114, filed on Nov. 11, 2015.

(51) Int. Cl. C12N 15/11; C12N 9/22

(52) U.S. Cl. CPC C12N 15/11; C12N 9/22; C12N 2710/10043; C12N 2750/14143; C12N 2740/15043; C12N 2310/20

(57) ABSTRACT

The present invention generally provides vectors, compositions, and methods of using the same for gene therapy.

### CROSS-REFERENCE TO RELATED APPLICATIONS

This application is a continuation of International Patent Application No. PCT/US2016/061633, filed Nov. 11, 2016, which claims the benefit of priority to U.S. Provisional Patent Application No. 62/254,114, filed Nov. 11, 2015, the contents of which are each incorporated herein by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SWCH_005_01US_SeqList_ST25.txt, date recorded: Apr. 24, 2018, file size 14 kilobytes).

### BACKGROUND

### Technical Field

The present invention generally relates to CRISPR-Cas systems, compositions, and related methods of use for gene therapy.

### Description of the Related Art

Channelopathies are a heterogeneous group of disorders resulting from the dysfunction of ion channels located in the membranes of all cells and many cellular organelles (see, e.g., Korean J Pediatrics 2014, 57(1): 1-18; Clin Neurophysiol. 2001, 112(1):2-18; Rev Neurol. 2001, 33(7):643-7). These include, for example, diseases of the nervous system (e.g., generalized epilepsy with febrile seizures plus, familial hemiplegic migraine, episodic ataxia, and hyperkalemic and hypokalemic periodic paralysis), the cardiovascular system (e.g., long QT syndrome, short QT syndrome, Brugada syndrome, and catecholaminergic polymorphic ventricular tachycardia), the respiratory system (e.g., cystic fibrosis), the endocrine system (e.g., neonatal diabetes mellitus, familial hyperinsulinemic hypoglycemia, thyrotoxic hypokalemic periodic paralysis, and familial hyperaldosteronism), the urinary system (e.g., Bartter syndrome, nephrogenic diabetes insipidus, autosomal-dominant polycystic kidney disease, and hypomagnesemia with secondary hypocalcemia), the immune system (e.g., myasthenia gravis, neuromyelitis optica, Isaac syndrome, and anti-NMDA [N-methyl-D-aspartate] receptor encephalitis), and others.

Channelopathies that primarily affect neurons include certain types of epilepsy, ataxia, migraine, hyperekplexia, blindness, deafness, and peripheral pain syndromes. Generalized epilepsy with febrile seizures plus (GEFS+) is a familial epilepsy syndrome that displays a broad spectrum of clinical phenotypes ranging from classical febrile seizures to Dravet syndrome (Brain Dev. 2009, 31:394-400). Dravet syndrome (also known as severe myoclonic epilepsy of infancy) is the most severe form that results from mutations in a voltage-gated sodium channel gene, SCN1A, or a γ-aminobutyric acid (GABA) receptor gene, GABRG2 (Neurobiol Dis. 2012, 48:115-123). Patients with Dravet syndrome suffer from refractory seizures, ataxia, and severe developmental delay with poor outcomes. The Nav1.1 channel, which is encoded by SCN1A, is one of nine a subtypes (Nav1.1-Nav1.9) of voltage-gated sodium channels and this subtype is preferentially expressed in GABAergic neurons. The GABAA receptor, which is encoded by GABRG2, is the major inhibitory neurotransmitter receptor in the central nervous system (CNS). Dysfunction of Nav1.1 channels or GABAA receptors can lead to reduced excitability of GABAergic neurons, thus resulting in brain hyperexcitability in patients with Dravet syndrome. Mutations in GABAA receptors have also been identified in other types of epilepsy, such as juvenile myoclonic epilepsy and childhood absence epilepsy (Ann Neurol. 2006, 59:983-987; J Physiol. 2011, 589:5857-5878; Am J Hum Genet. 2008, 82:1249-1261; J Neurosci. 2012, 32:5937-5952). Further, dysfunction of several voltage-gated sodium channels, including Nav1.3, Nav1.7, Nav1.8 and Nav1.9, have been shown to play specific roles in the neurobiology of pain (e.g., Muscle Nerve. 2012, 46(2): 155-165).

Unrelieved chronic pain is a critical health problem in the US and worldwide. A report by the Institute of Medicine estimated that 116 million Americans suffer from pain that persists for weeks to years, with resulting annual costs exceeding $560 million. There are no adequate long-term therapies for chronic pain sufferers, leading to significant cost for both society and the individual. Pain often results in disability and, even when not disabling, it has a profound effect on the quality of life. Pain treatment frequently fails even when the circumstances of care delivery are optimal, such as attentive, well-trained physicians; ready access to opioids; use of adjuvant analgesics; availability of patient-controlled analgesia; and evidence-based use of procedures like nerve blocks and IT pumps.

The most commonly used therapy for chronic pain is the application of opioid analgesics and nonsteroidal anti-inflammatory drugs, but these drugs can lead to addiction and may cause side effects, such as drug dependence, tolerance, respiratory depression, sedation, cognitive failure, hallucinations, and other systemic side effects. Despite the wide usage of pharmaceuticals, there is a strikingly low success rate for its effectiveness in pain relief. A large randomized study with various medications found only one out of every two or three patients achieving at least 50% pain relief (Finnerup et al., 2005). A follow-up study using the most developed pharmacological treatments found the same results, indicating that there was no improvement in the efficacy of medications for pain (Finnerup et al., 2010).

More invasive options for the treatment of pain include nerve blocks and electrical stimulation. A nerve block is a local anesthetic injection usually in the spinal cord to interrupt pain signals to the brain, the effect of which only lasts from weeks to months. Nerve blocks are not the recommended treatment option in most cases (Mailis and Taenzer, 2012). Electrical stimulation involves providing electric currents to block pain signals. Although the effect may last longer than a nerve block, complications arise with the electrical leads itself: dislocation, infection, breakage, or the battery dying. One review found that 40% of patients treated with electrical stimulation for neuropathy experienced one or more of these issues with the device (Wolter, 2014).

The most invasive, and least preferred, method for managing pain is complete surgical removal of the nerve or section thereof that is causing the pain. This option is only recommended when the patient has exhausted the former and other less invasive, treatments and found them ineffective. Radiofrequency nerve ablation uses heat to destroy problematic nerves and provides a longer pain relief than a nerve block. However, one study found no difference between the control and treatment groups in partial radiofrequency lesioning of the DRG for chronic lumbosacral radicular pain (Geurts et al., 2003). Other surgical methods for surgically removing the pain nerves suffer from similar shortcomings and have serious side effects long-term, including sensory or motor deficits, or cause pain elsewhere.

Further studies of the mechanisms of pain have opened the way for development of new treatment strategies, one of which is gene therapy. The key to gene therapy is selecting safe and highly efficient gene delivery systems that can deliver therapeutic genes to overexpress or suppress relevant targets in specific cell types.

However, few delivery systems have been shown to be safe and efficient; thus, the promise of gene therapy for treating channelopathies and/or managing pain has yet to be realized. The present invention addresses these needs and offers other related advantages.

### BRIEF SUMMARY

The present provides polynucleotides, CRISPR-Cas systems, polynucleotides, vectors, genetically modified cells, and related compositions for use in gene therapy.

In various embodiments, a nucleic acid comprising a CRISPR-Cas system and vectors comprising the same comprise an inducibly and transiently regulatable CRISPR-Cas system for use in the gene therapy. Without wishing to be bound by any particular theory, the present inventors have discovered that vectors contemplated herein provide several advantages compared to other gene therapy methods, including (1) efficient in vivo delivery; (2) delivery of a single vector comprising a complete gene therapy solution, including templates for gene correction/gene insertion; and (3) a regulatable genome editing platform with increased efficiency and reduced off-target effects.

In various embodiments, a nucleic acid comprising a CRISPR-Cas system for the treatment, prevention, or amelioration of a disease condition set forth in Table 2 or Table 3 is provided.

In various embodiments, a nucleic acid comprising a CRISPR-Cas system for the treatment, prevention, or amelioration of a channelopathy is provided.

In various more specific embodiments, a nucleic acid comprising a CRISPR-Cas system for the treatment, prevention, or amelioration of chronic pain is provided.

In various other embodiments, a nucleic acid disclosed herein comprises an inducibly and/or transiently regulatable CRISPR-Cas system.

In particular embodiments, the nucleic acid comprises: a first expression cassette that comprises an RNA polymerase II promoter operably linked to a polynucleotide encoding a CRISPR-Cas endonuclease; and a second expression cassette that comprises one or more RNA polymerase III promoters each operably linked to a polynucleotide encoding one or more guide RNAs.

In certain embodiments, the nucleic acid comprises: a first expression cassette that comprises at least one regulatory element for transient expression and an RNA polymerase II promoter operably linked to a polynucleotide encoding a CRISPR-Cas endonuclease; and a second expression cassette that comprises one or more RNA polymerase III promoters each operably linked to a polynucleotide encoding one or more guide RNAs.

In further embodiments, the nucleic acid comprises: a first expression cassette that comprises at least one regulatory element for inducible expression and at least one regulatory element for transient expression and a polynucleotide encoding a CRISPR-Cas endonuclease; a second expression cassette that comprises one or more RNA polymerase III promoters each operably linked to a polynucleotide encoding one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In particular embodiments, the nucleic acid comprises: a first expression cassette that comprises at least one regulatory element for transient expression and an RNA polymerase II promoter operably linked to a polynucleotide encoding a CRISPR-Cas endonuclease; a second expression cassette that comprises at least one regulatory element for inducible expression operably linked to a polynucleotide encoding one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In particular embodiments, the nucleic acid comprises: a first expression cassette that comprises at least one regulatory element for inducible expression and at least one regulatory element for transient expression and a polynucleotide encoding a CRISPR-Cas endonuclease; a second expression cassette that comprises one or more RNA polymerase III promoters each operably linked to one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In further embodiments, the at least one regulatory element for transient expression comprises one or more guide RNA target sites.

In some embodiments, the at least one regulatory element for transient expression comprises one or more guide RNA target sites and wherein the polynucleotide encoding the CRISPR-Cas endonuclease is flanked by the one or more guide RNA target sites.

In particular embodiments, the at least one regulatory element for transient expression comprises one or more guide RNA target sites and wherein the polynucleotide encoding the switch polypeptide is flanked by one or more guide RNA target sites.

In certain embodiments, the nucleic acid further comprises a polynucleotide encoding a template for altering at least one site in a genome that is flanked by one or more guide RNA target sites.

In additional embodiments, the guide RNA target sites flanking any one of the polynucleotide encoding the CRISPR-Cas endonuclease, the polynucleotide encoding the switch polypeptide, and the polynucleotide encoding the template for altering at least one site in the genome are the same.

In particular embodiments, the guide RNA target sites flanking the polynucleotide encoding the CRISPR-Cas endonuclease, the polynucleotide encoding the switch polypeptide, and the polynucleotide encoding the template for altering at least one site in the genome are the same.

In some embodiments, each of the guide RNA target sites flanking the 5′ end of the polynucleotide encoding the CRISPR-Cas endonuclease, the polynucleotide encoding the switch polypeptide, and the polynucleotide encoding the template for altering at least one site in the genome are the same.

In further embodiments, each of the guide RNA target site flanking the 3′ end of the polynucleotide encoding the CRISPR-Cas endonuclease, the polynucleotide encoding the switch polypeptide, and the polynucleotide encoding the template for altering at least one site in the genome are the same.

In particular embodiments, the guide RNA target site flanking the 5′ end and the guide RNA target site flanking the 3′ end of any one of the polynucleotides encoding the CRISPR-Cas endonuclease, the switch polypeptide, and the template for altering at least one site in the genome are different.

In certain embodiments, the guide RNA target site flanking the 5′ end and the guide RNA target site flanking the 3′ end of the polynucleotides encoding the CRISPR-Cas endonuclease, the switch polypeptide, and the template for altering at least one site in the genome are different.

In particular embodiments, each of the guide RNAs target sites flanking the 5′ end of the polynucleotides encoding the CRISPR-Cas endonuclease, the switch polypeptide, and the template for altering at least one site in the genome are the same; wherein each of the guide RNAs target sites flanking the 3′ end of the polynucleotides encoding the CRISPR-Cas endonuclease, the switch polypeptide, and the template for altering at least one site in the genome are the same; and wherein the guide RNA target site flanking the 5′ end each polynucleotide is different from the guide RNA target site flanking the 3′ end of each of polynucleotide.

In additional embodiments, the one or more guide RNA target sites in the vector are identical to one or more guide RNA target sites in the genome.

In some embodiments, the guide RNA target site flanking the 5′ end of each polynucleotide is identical to a guide RNA target site in the genome; wherein the guide RNA target site flanking the 3′ end of each polynucleotide is identical to a guide RNA target site in the genome; and wherein the guide RNA target site flanking the 5′ end each polynucleotide is different from the guide RNA target site flanking the 3′ end of each of polynucleotide.

In certain embodiments, the one or more guide RNAs recognize and bind to each of the one or more guide RNAs target sites contemplated herein.

In further embodiments, the vector comprises a single guide RNA that recognizes and binds all of the one or more guide RNA target sites contemplated herein.

In particular embodiments, the second expression cassette comprises a plurality of guide RNAs, wherein each of the plurality of guide RNAs recognizes and binds to one of the one or more guide RNA target sites contemplated herein.

In particular embodiments, at least one RNA polymerase II promoter is a ubiquitous promoter, optionally wherein each RNA polymerase II promoter is a ubiquitous promoter, optionally wherein each ubiquitous promoter is different.

In some embodiments, the switch polypeptide is selected from the group consisting of a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

In further embodiments, the ubiquitous promoter is independently selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In certain embodiments, the RNA polymerase II promoter is a tissue-specific or lineage-specific promoter.

Cis-Regulatory Elements (CREs e.g. promoters and enhancers) are DNA regions that may drive protein expression in a tissue or cell specific manner. CREs may be identified, isolated, and incorporated into gene therapy vehicles to selectively drive transgene expression in target cells. In particular embodiments, the tissue-specific or lineage-specific promoter is selected from the group consisting of: a neuron specific promoter, a promoter operable in a trigeminal ganglion (TGG) neuron, a dorsal root ganglion (DRG) neuron, an hSYN1 promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. CREs may be derived from endogenous ion channel proteins of which examples are included in Table 2 and Table 3.

In additional embodiments, at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

In some embodiments, the one or more RNA polymerase III promoters is selected from the group consisting of: a human U6 snRNA promoter, a mouse U6 snRNA promoter, a human H1 RNA promoter, a mouse H1 RNA promoter, and a human tRNA-val promoter.

In particular embodiments, the one or more RNA polymerase III promoters is independently selected from the group consisting of: a human U6 snRNA promoter, a mouse U6 snRNA promoter, a human H1 RNA promoter, a mouse H1 RNA promoter, and a human tRNA-val promoter.

In particular embodiments, the CRISPR-Cas endonuclease selected from the group consisting of: Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In particular embodiments, the CRISPR-Cas endonuclease comprises a Cas9 polypeptide.

In certain embodiments, the Cas9 polypeptide is isolated from Staphylococcus aureus, Streptococcus pyogenes, Streptococcus thermophilis, Treponema denticola, and Neisseria meningitidis.

In additional embodiments, the Cas9 polypeptide comprises one or more mutations in a HNH or a RuvC-like endonuclease domain or the HNH and the RuvC-like endonuclease domains.

In some embodiments, the mutant Cas9 polypeptide is a nickase.

In further embodiments, the mutant Cas9 polypeptide sequence is from Streptococcus pyogenes and comprises a mutation in the RuvC domain.

In certain embodiments, the mutation is a D10A mutation.

In particular embodiments, the mutant Cas9 polypeptide sequence is from Streptococcus pyogenes and comprises a mutation in the HNH domain.

In additional embodiments, the mutation is a D839A, H840A, or N863A mutation.

In some embodiments, the mutant Cas9 polypeptide sequence is from Streptococcus thermophilis and comprises a mutation in the RuvC-like domain.

In particular embodiments, the mutation is a D9A mutation.

In some embodiments, the mutant Cas9 polypeptide sequence is from Streptococcus thermophilis and comprises a mutation in the HNH domain.

In further embodiments, the mutation is a D598A, H599A, or N622A mutation.

In particular embodiments, the mutant Cas9 polypeptide sequence is from Treponema denticola and comprises a mutation in the RuvC-like domain.

In certain embodiments, the mutation is a D13A mutation.

In certain embodiments, the mutant Cas9 polypeptide sequence is from Treponema denticola and comprises a mutation in the HNH domain.

In certain embodiments, the mutation is a D878A, H879A, or N902A mutation.

In additional embodiments, the mutant Cas9 polypeptide sequence is from Neisseria meningitidis and comprises a mutation in the RuvC domain.

In some embodiments, the mutation is a D16A mutation.

In certain embodiments, the mutant Cas9 polypeptide sequence is from Neisseria meningitidis and comprises a mutation in the HNH domain.

In particular embodiments, the mutation is a D587A, H588A, or N611A mutation.

In some embodiments, the mutant Cas9 polypeptide sequence is from Staphylococcus aureus and comprises a mutation in the RuvC domain.

In further embodiments, the mutation is a D10A mutation.

In further embodiments, the mutant Cas9 polypeptide sequence is from Staphylococcus aureus and comprises a mutation in the HNH domain.

In particular embodiments, the mutation is a N580A mutation.

In some embodiments, the Cas9 is a human codon optimized Cas9.

In particular embodiments, the CRISPR-Cas endonuclease is a Cpf1 polypeptide.

In additional embodiments, the first expression cassette comprises a polynucleotide encoding a Cpf1 polypeptide isolated from Francisella novicida, Acidaminococcus sp. BV3L6, or Lachnospiraceae bacterium ND2006.

In certain embodiments, the Cpf1 polypeptide comprises one or more mutations in a RuvC-like endonuclease domain.

In some embodiments, the mutant Cpf1 polypeptide sequence is from Francisella novicida and comprises a mutation in the RuvC-like domain.

In additional embodiments, the mutation is a D917A, E1006A, or D1225A mutation.

In particular embodiments, the CRISPR-Cas endonuclease is a Cas9 fusion polypeptide or a Cpf1 fusion polypeptide.

In particular embodiments, the fusion polypeptide comprises one or more functional domains.

In particular embodiments, the one or more functional domains is selected from the group consisting of: a histone methylase or demethylase domains, a histone acetylase or deacetylase domains, a SUMOylation domain, an ubiquitylation or deubiquitylation domain, a DNA methylase or DNA demethylase domain, and a nuclease domain.

In further embodiments, the nuclease domain is a FOK I nuclease domain.

In some embodiments, the nuclease domain is a TREX2 nuclease domain.

In certain embodiments, the switch polypeptide comprises a TREX2 domain or is a polypeptide comprising a self-cleaving viral peptide and TREX2.

In additional embodiments, the one or more guide RNAs are single strand guide RNAS (sgRNAs).

In particular embodiments, the one or more guide RNAs are crRNAs.

In some embodiments, the polynucleotide encoding the CRISPR-Cas endonuclease further encodes an inhibitory RNA and a binding site for the inhibitory RNA.

In some embodiments, the inhibitory RNA is a miRNA or a mishRNA.

In particular embodiments, the polynucleotide encoding the CRISPR-Cas endonuclease further comprises an intron, wherein the intron is spliced in mammalian cells but not in non-mammalian cells.

In additional embodiments, the intron is an artificial intron.

In further embodiments, the intron is a human growth hormone intron.

In some embodiments, the intron is an SV40 large T-antigen intron.

In particular embodiments, the intron is an intron isolated from a mammalian gene.

In certain embodiments, one or more guide RNAs are design to alter at least one site in a genome.

In additional embodiments, the at least one site in the genome is in a gene set forth in Table 3, or a gene associated with a disease set forth in Table 2 or Table 3.

In additional embodiments, the at least one site in the genome is in a gene associated with a human channelopathy.

In additional embodiments, at least one site in the genome is in a gene associated with the signaling of pain.

In some embodiments, the at least one site in the genome is in a gene encoding a voltage gated sodium channel.

In certain embodiments, the voltage gated sodium channel is selected from the group consisting of: Naᵥ1.1, Naᵥ1.2, Naᵥ1.3, Naᵥ1.5, Naᵥ1.6, Naᵥ1.7, Naᵥ1.8, and Naᵥ1.9.

In particular embodiments, the sequence of the one or more guide RNAs is selected from the group consisting of SEQ ID NOs: 1-55.

In additional embodiments, the nucleic acid further comprises a polynucleotide encoding a template for altering at least one site in a genome.

In certain embodiments, the template comprises a regulatable transcriptional regulatory element.

In certain embodiments, the transcriptional regulatory element is targeted for insertion upstream of a transcription start site in a gene of the cell.

In additional embodiments, the transcriptional regulatory element is activated in the presence of an exogenous ligand or small molecule.

In further embodiments, the transcriptional regulatory element is activated in the absence of an exogenous ligand or small molecule.

In particular embodiments, the transcriptional regulatory element is repressed in the presence of an exogenous ligand or small molecule.

In additional embodiments, the transcriptional regulatory element is repressed in the absence of an exogenous ligand or small molecule.

In some embodiments, the transcriptional regulatory element is inserted upstream of a gene set forth in Table 3, or a gene associated with a disease set forth in Table 2 or Table 3.

In some embodiments, the transcriptional regulatory element is inserted upstream of a gene associated with a human channelopathy.

In some embodiments, the transcriptional regulatory element is inserted upstream of a gene associated with the signaling of pain.

In certain embodiments, the transcriptional regulatory element is inserted upstream of a gene encoding a voltage gated channel, such as a voltage gated sodium or potassium channel.

In some embodiments, the voltage gated sodium channel is selected from the group consisting of: Naᵥ1.1, Naᵥ1.2, Naᵥ1.3, Naᵥ1.5, Naᵥ1.6, Naᵥ1.7, Naᵥ1.8, and Naᵥ1.9.

In particular embodiments, the nucleic acid further comprises an epitope tag.

In additional embodiments, the epitope tag is selected from the group consisting of: maltose binding protein (“MBP”), glutathione S transferase (GST), HIS6, MYC, FLAG, V5, VSV-G, and HA.

In further embodiments, the nucleic acid further comprises one or more poly(A) sequences.

In certain embodiments, the one or more poly(A) sequences are selected from the group consisting of: an artifical poly(A) sequence, an SV40 poly(A) sequence, a bovine growth hormone poly(A) sequence (bGHpA), and a rabbit β-globin poly(A) sequence (rβgpA).

In various embodiments, a vector comprising the nucleic acid of any one of claims or as shown in any one of the figures or embodiments disclosed or contemplated herein is provided.

In certain embodiments, a viral vector comprising a nucleic acid contemplated herein is provided.

In certain embodiments, an adenoviral vector comprising a nucleic acid contemplated herein is provided.

In particular embodiments, a lentiviral vector comprising a nucleic acid contemplated herein is provided.

In additional embodiments, the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

In particular embodiments, the vector comprises a chimeric 5′ LTR.

In particular embodiments, the vector comprises a 3′ self-inactivating (SIN) LTR.

In some embodiments, the vector comprises a cPPT/FLAP sequence.

In further embodiments, the vector comprises a woodchuck post-transcriptional regulatory element (WPRE).

In various embodiments, an adenoviral-associated virus (AAV) vector comprising a nucleic acid contemplated herein is provided.

In particular embodiments, the AAV vector comprises one or more AAV2 inverted terminal repeats (ITRs).

In certain embodiments, the AAV vector comprises a serotype selected from the group consisting of: AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In additional embodiments, the AAV vector comprises a serotype selected from the group consisting of: AAV1, AAV5, AAV6, AAV6 (Y705F/Y731F/T492V), AAV8, AAV9, and AAV9 (Y731F).

In particular embodiments, the AAV vector comprises a serotype selected from the group consisting of: AAV6, AAV6 (Y705F/Y731F/T492V), AAV9, and AAV9 (Y731F).

In particular embodiments, the AAV vector comprises an AAV6 or AAV6 (Y705F/Y731F/T492V) serotype.

In certain embodiments, the AAV vector is a self-complementary AAV (scAAV) vector.

In various embodiments, a composition comprising a nucleic acid contemplated herein and optionally, one or more exosomes, nanoparticles, or biolistics is provided.

In certain various embodiments, a composition comprising a vector contemplated herein is provided.

In other various embodiments, a method of managing, preventing, or treating pain in a subject, comprising administering to the subject a composition contemplated herein is provided.

In various particular embodiments, a method of providing analgesia to a subject having pain, comprising administering to the subject the composition contemplated herein is provided.

In particular embodiments, the pain is acute pain or chronic pain.

In certain embodiments, the pain is chronic pain.

In certain embodiments, the pain is acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, or menstrual pain.

In further embodiments, the pain is nociceptive pain.

In additional embodiments, the pain is nociceptive pain is selected from the group consisting of central nervous system trauma, strains/sprains, burns, myocardial infarction and acute pancreatitis, post-operative pain (pain following any type of surgical procedure), posttraumatic pain, renal colic, cancer pain and back pain.

In some embodiments, the pain is neuropathic pain.

In some embodiments, the etiology of the neuropathic pain is selected from the group consisting of: peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

In particular embodiments, the neuropathic pain is related to a pain disorder selected from the group consisting of: arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

In additional embodiments, the pain is inflammatory pain.

In particular embodiments, the pain is associated with musculoskeletal disorders, myalgia, fibromyalgia, spondylitis, sero-negative (non-rheumatoid) arthropathies, non-articular rheumatism, dystrophinopathy, glycogenolysis, polymyositis and pyomyositis; heart and vascular pain, pain caused by angina, myocardical infarction, mitral stenosis, pericarditis, Raynaud's phenomenon, scleredoma and skeletal muscle ischemia; head pain, migraine, cluster headache, tension-type headache mixed headache and headache associated with vascular disorders; orofacial pain, dental pain, otic pain, burning mouth syndrome, and temporomandibular myofascial pain.

In various embodiments, a method of treating, preventing, ameliorating, or managing a disease set forth in Table 2 or Table 3 in a subject comprising administering a nucleic acid, vector, or composition contemplated herein to an appropriate cell type (e.g., a neuronal or other cell type) of the subject is provided.

In various embodiments, a method of treating, preventing, ameliorating, or managing a channelopathy in a subject comprising administering a nucleic acid, vector, or composition contemplated herein to one or more neuronal cells of the subject is provided.

In particular embodiments, the channelopathy is associated with a mutation in a voltage gated ion channel, such as a voltage gated sodium or potassium channel.

In certain embodiments, the voltage gated sodium channel is selected from the group consisting of: Naᵥ1.1, Naᵥ1.2, Naᵥ1.3, Naᵥ1.4, Naᵥ1.5, Naᵥ1.6, Naᵥ1.7, Naᵥ1.8, and Naᵥ1.9.

In further embodiments, the channelopathy is selected from the group consisting of: Channelopathy-associated Insensitivity to Pain (CIP), Primary Erythermalgia (PE), Fibromyalgia, Paroxysmal Extreme Pain Disorder (PEPD), Febrile Epilepsy, Generalized Epilepsy with Febrile Seizures, Dravet syndrome, West syndrome, Doose syndrome, Intractable Childhood Epilepsy with Generalized Tonic-Clonic seizures (ICEGTC), Panayiotopoulos syndrome, Familial Hemiplegic Migraine (FHM), Familial Autism, Rasmussen's Encephalitis, Lennox-Gastaut syndrome, Epilepsy, Pain, Hyperkalemic Periodic Paralysis, Paramyotonia Congenita, Potassium-Aggravated Myotonia, Long QT Syndrome, Brugada Syndrome, Idiopathic Ventricular Fibrillation, Irritable Bowel Syndrome, Neuropsychiatric Disorders, or any other channelopathy known in the art and/or set forth in Table 2 or Table 3.

In further embodiments, a nucleic acid, vector, or composition contemplated herein is intrathecally administered to a subject.

In additional embodiments, a nucleic acid, vector, or composition contemplated herein is intraganglionicly administered to a subject.

In some embodiments, a nucleic acid, vector, or composition contemplated herein is intraneurally administered to a subject.

In particular embodiments, a nucleic acid, vector, or composition contemplated herein is intramuscularly administered to a subject.

In certain embodiments, a nucleic acid, vector, or composition contemplated herein is intracranially administered to a subject.

In particular embodiments, a nucleic acid, vector, or composition contemplated herein is administered to a subject by electroporation.

### BRIEF DESCRIPTION OF THE SEVERAL VIEWS OF THE DRAWINGS

FIG. 1 shows a diagram of an AAV vector for the delivery of a CRISPR-Cas genome editing platform for genome modification.

FIG. 2 shows a diagram of an AAV vector for the delivery of a CRISPR-Cas self-regulating genome editing platform for genome modification.

FIG. 3 shows a diagram of an AAV vector for the delivery of a CRISPR-Cas self-regulating genome editing platform for genome modification.

FIG. 4 shows a diagram of an AAV vector for the delivery of a CRISPR-Cas self-regulating genome editing platform for genome modification.

FIG. 5 shows a diagram of an AAV vector for the delivery of a CRISPR-Cas self-regulating genome editing platform for genome modification.

FIG. 6 shows a diagram of an AAV vector for the delivery of a CRISPR-Cas self-regulating genome editing platform for genome modification.

### BRIEF DESCRIPTION OF THE SEQUENCE IDENTIFIERS

SEQ ID NOs: 1-55 set forth the polynucleotide sequences of exemplary sgRNAs for the treatment, prevention or amelioration of pain in a subject.

### DETAILED DESCRIPTION

### A. Overview

The present invention generally relates to gene therapy compositions and methods that provide efficient delivery in vitro, ex vivo, or in vivo, that are engineered to provide a safe and reliable genome editing platform, and that offer precise spatiotemporal control over particular cell types associated with diseases or other conditions, such as neuronal cells involved in the pain pathway. The present invention offers these and other related advantages compared to existing therapies.

In various embodiments, a polynucleiotide comprising an inducibly and transiently regulatable genome editing platform for the disruption, deletion, correction, or insertion of genetic material at a genome sequence is provided. The polynucleotides contemplated herein comprise a CRISPR-Cas genome editing platform that has been modified to enhance both safety and efficacy of the genome editing. Without wishing to be bound to a particular theory, the polynucleotides provide the advantage of delivering a complete genome editing platform in a single polynucleotide and also provide a more efficient genome editing platform that is less prone to off-target effects. The genome editing platform is less prone to off-target effects, in part, because the polynucleotides contemplated herein provide inducibly and/or transiently regulatable CRISPR-Cas endonucleases.

In various embodiments, a viral vector comprising a CRISPR-Cas genome editing platform is administered to a subject to treat, prevent or ameliorate the symptoms or effects of a disease set forth in Table 2 or Table 3.

In various embodiments, a viral vector comprising a CRISPR-Cas genome editing platform is administered to a subject to treat, prevent or ameliorate the symptoms or effects of a human channelopathy.

In various more specific embodiments, a viral vector comprising a CRISPR-Cas genome editing platform is administered to a subject to safely and efficiently manage pain.

The vectors and compositions contemplated herein are used to attenuate the sensation of pain in a subject. In various embodiments, the pain is acute pain or chronic pain. The chronic pain can be nociceptive pain or neuropathic pain. In one embodiment, the pain is neuropathic pain. The pain can also be an isolated pain, or the pain can be associated with a particular disease.

Accordingly, the present invention addresses an unmet clinical need for improving the safety and efficacy of gene therapy in pain management.

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

The practice of the present invention will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al., Molecular Cloning: A Laboratory Manual (2nd Edition, 1989); Maniatis et al., Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); A Practical Guide to Molecular Cloning (B. Perbal, ed., 1984).

All publications, patents and patent applications cited herein are hereby incorporated by reference in their entirety.

### B. Definitions

Unless defined otherwise, all technical and scientific terms used herein have the same meaning as commonly understood by those of ordinary skill in the art to which the invention belongs. For the purposes of the present invention, the following terms are defined below.

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “isolated” means material that is substantially or essentially free from components that normally accompany it in its native state. In particular embodiments, the term “obtained” or “derived” is used synonymously with isolated.

A “subject,” or “individual” as used herein, includes any animal that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect associated with a reduction in one or more symptoms or other effects of a disease or condition disclosed herein, such as a disease or condition set forth in Table 2 or Table 3. For example, in relation to embodiments comprising the treatment of pain in a subject, “treatment” or “treating,” includes any beneficial or desirable effect associated with a reduction in pain, and may include even minimal reductions in pain. Treatment can involve optionally either the reduction or amelioration of pain, or the delaying of the progression of pain. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of one or more symptoms or other effects of a disease or condition disclosed herein, such as a disease or condition set forth in Table 2 or Table 3. For example, in embodiments that relate to treating pain in a subject, “prevent,” and similar words such as “prevented,” “preventing” etc., indicate an approach for preventing, inhibiting, or reducing the likelihood of the occurrence or recurrence of pain. It also refers to delaying the onset or recurrence of a disease or condition or delaying the occurrence or recurrence of the symptoms of pain. As used herein, “prevention” and similar words also includes reducing the intensity, effect, symptoms and/or burden of pain prior to onset or recurrence.

As used herein, “management” or “controlling” one or more symptoms or effects of a disease or condition (e.g., pain) refers to the use of the compositions or methods contemplated herein, to improve the quality of life for an individual by providing relief in one form or another to the patient, e.g., by providing analgesia to a subject suffering from pain.

As used herein, the term “amount” refers to “an amount effective” or “an effective amount” of a composition, polynucleotide, or viral vector contemplated herein sufficient to achieve a beneficial or desired prophylactic or therapeutic result, including clinical results.

A “prophylactically effective amount” refers to an amount of a composition, polynucleotide, or viral vector contemplated herein sufficient to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus may vary according to factors such as the disease state, age, sex, and weight of the individual, and the ability of the stem and progenitor cells to elicit a desired response in the individual. A therapeutically effective amount is also one in which any toxic or detrimental effects of a composition, polynucleotide, or viral vector contemplated herein are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated cell.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

In some embodiments, the compositions, polynucleotides, and vectors are administered to an “excitable cell.” As used herein, the term “excitable cell” refers to a cell that experiences fluctuations in its membrane potential as a result of gated ion channels. Illustrative examples of excitable cells contemplated herein include but are not limited to myocytes, neuronal cells, and the like.

In particular embodiments, the neuronal cell is a sensory neuron. Illustrative examples of sensory neurons include, but are not limited to, dorsal root ganglion (DRG) neurons and trigeminal ganglion (TGG) neurons. In one embodiment, the neuronal cell is a peripheral sensory neuron. In one embodiment, the neuronal cell is an inhibitory interneuron.

### C. Polynucleotides

In various illustrative embodiments, the present invention contemplates, in part, polynucleotides, nucleic acids, polynucleotides encoding polypeptides and fusion polypeptides, viral vector polynucleotides, that reconsitute entire genome editing platforms and compositions comprising the same.

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded. Polynucleotides include, but are not limited to: premessenger RNA (pre-mRNA), messenger RNA (mRNA), RNA, short interfering RNA (siRNA), short hairpin RNA (shRNA), microRNA (miRNA), ribozymes, synthetic RNA, genomic RNA (geRNA), guide RNA, tracRNA, crRNA, sgRNA, plus strand RNA (RNA(+)), minus strand RNA (RNA(−)), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxynucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence described herein or known in the art, typically where the variant maintains at least one biological activity of the reference sequence.

In one embodiment, a nucleic acid comprises a plurality of expression cassettes. The expression cassette comprises an expression control sequence operably linked to a polynucleotide. The polynucleotide may be a gene or a cDNA encoding a protein or a polynucleotide encoding an inhibitory RNA or an RNA sequence that is required for genome editing in a CRISPR-Cas system contemplated herein, e.g., a guide RNA, a tracRNA, crRNA, or sgRNA.

As used herein, the term “gene” may refer to a polynucleotide sequence comprising enhancers, promoters, introns, exons, and the like. In particular embodiments, the term “gene” refers to a polynucleotide sequence encoding a polypeptide, regardless of whether the polynucleotide sequence is identical to the genomic sequence encoding the polypeptide. In particular embodiments, the term “gene” refers to a cDNA.

A “genomic sequence regulating transcription of” or a “genomic sequence that regulates transcription or” refers to a polynucleotide sequence that is associated with the transcription of a gene. In one embodiment, the genomic sequence regulates transcription because it is a binding site for a polypeptide that represses or decreases transcription or a polynucleotide sequence associated with a transcription factor binding site that contributes to transcriptional repression.

A “cis-acting sequence regulating transcription of” or a “cis-acting nucleotide sequence that regulates transcription or” or equivalents refers to a polynucleotide sequence that is associated with the transcription of a gene. In one embodiment, the cis-acting sequence regulates transcription because it is a binding site for a polypeptide that represses or decreases transcription or a polynucleotide sequence associated with a transcription factor binding site that contributes to transcriptional repression.

A “regulatory element” or “cis-acting sequence” or “transcriptional regulatory element” or equivalents thereof refer to an expression control sequence that comprises a polynucleotide sequence that is associated with the transcription or expression of a polynucleotide sequence encoding a polypeptide.

A “regulatory element for inducible expression” refers to a polynucleotide sequence that is a promoter, enhancer, or functional fragment thereof that is operably linked to a polynucleotide to be expressed. The regulatory element for inducible expression responds to the presence or absence of a molecule that binds the element to increase (turn-on) or decrease (turn-off) the expression of the polynucleotide operably linked thereto. Illustrative regulatory elements for inducible expression include, but are not limited to, a tetracycline responsive promoter, an ecdysone responsive promoter, a cumate responsive promoter, a glucocorticoid responsive promoter, an estrogen responsive promoter, an RU-486 responsive promoter, a peroxisome proliferator-activated receptor-gamma (PPAR-γ) promoter, and a peroxide inducible promoter.

A “regulatory element for transient expression” refers to a polynucleotide sequence that can be used to briefly or temporarily express a polynucleotide nucleotide sequence. In particular embodiments, one or more regulatory elements for transient expression can be used to limit the duration of polynucleotide expression. In certain embodiments, the preferred duration of polynucleotide expression is on the order of minutes, hours, or days. Illustrative regulatory elements for transient expression include, but are not limited to, nuclease target sites, recombinase recognition sites, and inhibitory RNA target sites. In addition, to some extent, in particular embodiments, a regulatory element for inducible expression may also contribute to controlling the duration of polynucleotide expression.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

In one embodiment, a polynucleotide comprises a nucleotide sequence that hybridizes to a target nucleic acid sequence under stringent conditions. To hybridize under “stringent conditions” describes hybridization protocols in which nucleotide sequences at least 60% identical to each other remain hybridized. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Terms used to describe sequence relationships between two or more polynucleotides or polypeptides include “reference sequence,” “comparison window,” “sequence identity,” “percentage of sequence identity,” and “substantial identity.” A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc, 1994-1998, Chapter 15.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant DNA, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The term “flanked” refers to a polynucleotide sequence that is adjacent to another sequence or that is in between an upstream polynucleotide sequence and/or a downstream poylnucleotide sequence, i.e., 5′ and/or 3′, relative to the sequence. For example, a sequence that is “flanked” by two other elements, indicates that one element is located 5′ to the sequence and the other is located 3′ to the sequence; however, there may be intervening sequences therebetween.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The terms “nucleic acid cassette” or “expression cassette” as used herein refers to polynucleotide sequences within a larger polynucleotide, such as a vector, which are sufficient to express one or more RNAs from a polynucleotide. The expressed RNAs may be translated into proteins, may function as guide RNAs or inhibitory RNAs to target other polynucleotide sequences for cleavage and/or degradation. In one embodiment, the nucleic acid cassette contains one or more polynucleotide(s)-of-interest. In another embodiment, the nucleic acid cassette contains one or more expression control sequences operably linked to one or more polynucleotide(s)-of-interest. Polynucleotides include polynucleotide(s)-of-interest. As used herein, the term “polynucleotide-of-interest” refers to a polynucleotide encoding a polypeptide or fusion polypeptide or a polynucleotide that serves as a template for the transcription of an inhibitory polynucleotide, e.g., guide RNA or inhibitory RNA, as contemplated herein. In a particular embodiment, a polynucleotide-of-interest encodes a polypeptide or fusion polypeptide having one or more enzymatic activities, such as a nuclease activity and/or chromatin remodeling or epigenetic modification activities.

Vectors may comprise 1, 2, 3, 4, 5, 6, 7, 8, 9 or 10 or more nucleic acid cassettes. In a preferred embodiment of the invention, a nucleic acid cassette comprises one or more expression control sequences operably linked to a component of a genome editing platform for gene therapy. The cassette can be removed from or inserted into other polynucleotide sequences, e.g., a plasmid or viral vector, as a single unit.

In one embodiment, a polynucleotide contemplated herein comprises 1, 2, 3, 4, 5, 6, 7, 8, 9, or more nucleic acid cassettes any number or combination of which may be in the same or opposite orientations.

Moreover, it will be appreciated by those of ordinary skill in the art that, as a result of the degeneracy of the genetic code, there are many nucleotide sequences that may encode a polypeptide, or fragment of variant thereof, as contemplated herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. In one embodiment, polynucleotides comprising particular allelic sequences are provided. Alleles are endogenous polynucleotide sequences that are altered as a result of one or more mutations, such as deletions, additions and/or substitutions of nucleotides.

In a certain embodiment, a polynucleotide-of-interest encodes an inhibitory polynucleotide including, but not limited to, a crRNA, a tracrRNA, a single guide RNA (sgRNA), an siRNA, an miRNA, an shRNA, a ribozyme or another inhibitory RNA.

In one embodiment, a polynucleotide-of-interest comprises a crRNA, a tracrRNA, or a single guide RNA (sgRNA). These RNAs are part of the CRISPR (Clustered Regularly Interspaced Short Palindromic Repeats)/Cas (CRISPR Associated) nuclease system; a recently engineered nuclease system based on a bacterial system that can be used for mammalian genome engineering. See, e.g., Jinek et al. (2012) Science 337:816-821; Cong et al. (2013) Science 339:819-823; Mali et al. (2013) Science 339:823-826; Qi et al. (2013) Cell 152:1173-1183; Jinek et al. (2013), eLife 2:e00471; David Segal (2013) eLife 2:e00563; Ran et al. (2013) Nature Protocols 8(11):2281-2308; Zetsche et al. (2015) Cell 163(3):759-771; PCT Pub. Nos.: WO2007025097; WO2008021207; WO2010011961; WO2010054108; WO2010054154; WO2012054726; WO2012149470; WO2012164565; WO2013098244; WO2013126794; WO2013141680; WO2013142578; U.S. Pat. App. Pub. Nos: US20100093617; US20130011828; US20100257638; US20100076057; US20110217739; US20110300538; US20130288251; US20120277120; and U.S. Pat. No. 8,546,553, each of which is incorporated herein by reference in its entirety.

In particular embodiments, the polynucleotide-of-interest is an inhibitory RNA that targets a gene set forth in Table 2 or Table 3 or a gene associated with a disease set forth in Table 2 or Table 3.

In particular embodiments, the polynucleotide-of-interest is an inhibitory RNA that targets a gene associated with a channelopathy.

In particular embodiments, the polynucleotide-of-interest is an inhibitory RNA that targets a molecule that is associated with the sensation and signaling of pain, e.g., TNFα, Nav1.1, Nav1.3, Nav1.6, Nav1.7, Nav1.8, Nav1.9, TRPV1, TRPV2, TRPV3, TRPV4, TRPC, TRPP, ACCN1, ACCN2, TRPM8, TRPA1, P2XR3, P2RY, BDKRB1, BDKRB2, Htr3A, ACCNs, KCNQ, HCN2, HCN4, CSF-1, CACNA1A-S, CACNA2D1, IL1, IL6, IL12, IL18, COX-2, NTRK1, NGF, GDNF, LIF, CCL2, CNR2, TLR2, TLR4, P2RX4, P2RX7, CCL2, CX3CR1, and BDNF.

Multiple class 1 CRISPR-Cas systems, which include the type I and type III systems, have been identified and functionally characterized in detail, revealing the complex architecture and dynamics of the effector complexes (Brouns et al., 2008, Marraffini and Sontheimer, 2008, Hale et al., 2009, Sinkunas et al., 2013, Jackson et al., 2014, Mulepati et al., 2014). In addition, several class 2-type II CRISPR-Cas systems that employ homologous RNA-guided endonucleases of the Cas9 family as effectors have also been identified and experimentally characterized (Barrangou et al., 2007, Garneau et al., 2010, Deltcheva et al., 2011, Sapranauskas et al., 2011, Jinek et al., 2012, Gasiunas et al., 2012). A second, putative class 2-type V CRISPR-Cas system has been recently identified in several bacterial genomes. The putative type V CRISPR-Cas systems contain a large, ˜1,300 amino acid protein called Cpf1 (CRISPR from Prevotella and Francisella 1).

The CRISPR/Cas nuclease system can be used to introduce a double-strand break in a target polynucleotide sequence, which may be repaired by non-homologous end joining (NHEJ) in the absence of a polynucleotide template, e.g., a DNA template for altering at least one site in a genome, or by homology directed repair (HDR), i.e., homologous recombination, in the presence of a polynucleotide repair template. Cas9 and Cpf1 nucleases can also be engineered as nickases, which generate single-stranded DNA breaks that can be repaired using the cell's base-excision-repair (BER) machinery or homologous recombination in the presence of a repair template. NHEJ is an error-prone process that frequently results in the formation of small insertions and deletions that disrupt gene function. Homologous recombination requires homologous DNA as a template for repair and can be leveraged to create a limitless variety of modifications specified by the introduction of donor DNA containing the desired sequence flanked on either side by sequences bearing homology to the target.

In various embodiments, vectors contemplated herein contain polynucleotides to be expressed that are flanked by one or more crRNA or sgRNA target sites to transiently regulate the expression of the polynucleotide.

In one embodiment, wherein a crRNA or sgRNA is directed against a polynucleotide sequence encoding a polypeptide, NHEJ of the ends of the cleaved genomic sequence may result in a normal polypeptide, a loss-of- or gain-of-function polypeptide, or knock-out of a functional polypeptide.

In another embodiment, wherein a crRNA or sgRNA is directed against a polynucleotide sequence encoding a cis-acting sequence that regulates mRNA expression of a polynucleotide sequence encoding a polypeptide, NHEJ of the genomic sequence may result increased expression, decreased expression, or complete loss of expression of the mRNA and polypeptide.

In another embodiment, wherein a polynucleotide template for repair of the cleaved genomic sequence is provided, the genomic locus is repaired with the sequence of the template by homologous recombination. In one embodiment, the repair template comprises a polynucleotide sequence that is different from a targeted genomic sequence. In one embodiment, the repair template comprises one or more polynucleotides that restores function of the targeted genomic sequence or restores the natural polynucleotide sequence encoding a wild type allele of a polypeptide. In another embodiment, the repair DNA template comprises one or more polynucleotides that reduces or eliminates function of the targeted genomic sequence or decreases the expression of the natural polynucleotide sequence encoding a wild type allele of a polypeptide and/or increasing the expression of a variant polypeptide. In another embodiment, the repair DNA template comprises one or more expression control sequences or transcription regulatory sequences that regulates the transcriptional activity of the locus.

As used herein, the term “guide RNA” refers to a “crRNA” and/or an “sgRNA.”

As used herein, the term “crRNA” refers to an RNA comprising a region of partial or total complementarity referred to herein as a “spacer motif” to a target polynucleotide sequence referred to herein as a protospacer motif. In one embodiment, a protospacer motif is a 20 nucleotide target sequence. In particular embodiments, the protospacer motif is 18, 19, 20, 21, 22, 23, 24, 25, 26, 27, 28, 29, 30, or more nucleotides. Without wishing to be bound by any particular theory, it is contemplated that protospacer target sequences of various lengths will be recognized by different bacterial species.

In one embodiment, the region of complementarity comprises a polynucleotide sequence that is at least 80%, 81%, 82%, 83%, 84%, 85%, 86%, 87%, 88%, 89%, 90%, 91%, 92%, 93%, 94%, 95%, 96%, 97%, 98%, 99% or 100% identical to the protospacer sequence. In a related embodiment, at least 10, 11, 12, 13, 14, 15, 16, 17, 18, 19, or 20 or more polynucleotides in the region of complementarity are identical to the protospacer motif. In a preferred embodiment, at least 10 of the 3′ most sequence in the protospacer motif is complementary to the crRNA sequence.

As used herein, the term “tracrRNA” refers to a trans-activating RNA that associates with the crRNA sequence through a region of partial complementarity and serves to recruit a Cas9 nuclease to the protospacer motif. In one embodiment, the tracrRNA is at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length. In one embodiment, the tracrRNA is about 85 nucleotides in length.

In one embodiment, the crRNA and tracrRNA are engineered into one polynucleotide sequence referred to herein as a “single guide RNA” or “sgRNA.” The crRNA equivalent portion of the sgRNA is engineered to guide the Cas9 nuclease to target any desired protospacer motif. In one embodiment, the tracrRNA equivalent portion of the sgRNA is engineered to be at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more nucleotides in length.

Certain illustrative examples of sgRNAs suitable for use in particular embodiments contemplated herein, particularly in relation to treating, preventing or ameliorating the symptoms of pain in a subject, include, but are not limited, to sgRNA sequences as set forth in SEQ ID NOs: 1-55. Of course, it will be understood by the skilled artisan, in light of the present disclosure and the level and nature of skill in the art, how to design, prepare and implement further sgRNA sequences of interest in relation to other diseases or conditions disclosed herein, such as the diseases or conditions set forth in Table 2 or Table 3.

The protospacer motif abuts a short protospacer adjacent motif (PAM), which plays a role in recruiting a Cas9/RNA or Cpf1/RNAcomplex. Cas9 polypeptides recognize PAM motifs specific to the Cas9 polypeptide. Accordingly, the CRISPR/Cas9 system can be used to target and cleave either or both strands of a double-stranded polynucleotide sequence flanked by particular 3′ PAM sequences specific to a particular Cas9 polypeptide. PAMs may be identified using bioinformatics or using experimental approaches. Esvelt et al., 2013, Nature Methods. 10(11):1116-1121, which is hereby incorporated by reference in its entirety.

In one embodiment, a polynucleotide encodes a transiently regulatable Cas9 polypeptide. In one embodiment, the polynucleotide comprises a regulatory element for transient expression of and a polynucleotide encoding a Cas9 polypeptide. A Cas9 polypeptide can be engineered as a double-stranded DNA endonuclease or a nickase or catalytically dead Cas9, and forms a ternary target complex with a crRNA and a tracrRNA for site specific DNA recognition and cleavage if catalytically active. Normally, tracrRNA is involved in the maturation of precursor crRNA. Following co-processing of tracrRNA and pre-crRNA by RNase III, a dual-tracrRNA:crRNA guides the CRISPR-associated endonuclease Cas9 to site-specifically cleave a target DNA, e.g., protospacer sequence.

Unlike Cas9 systems, Cpf1-containing CRISPR-Cas systems have three features. First, Cpf1-associated CRISPR arrays are processed into mature crRNAs without the requirement of an additional trans-activating crRNA (tracrRNA) (Deltcheva et al., 2011, Chylinski et al., 2013). Second, Cpf1-crRNA complexes efficiently cleave target DNA proceeded by a short T-rich protospacer-adjacent motif (5′-TTN PAM), in contrast to the G-rich PAM following the target DNA for Cas9 systems. Third, Cpf1 introduces a staggered DNA double-stranded break with a 4 or 5-nt 5′ overhang.

In one embodiment, a polynucleotide encodes a transiently regulatable Cpf1 polypeptide. In one embodiment, the polynucleotide comprises a regulatory element for transient expression of and a polynucleotide encoding a Cpf1 polypeptide. A Cpf1 polypeptide can be engineered as a double-stranded DNA endonuclease or a nickase or catalytically dead Cpf1, and forms a target complex with a crRNA for site specific DNA recognition and cleavage if catalytically active. Following processing of pre-crRNA by RNase III, a crRNA guides the CRISPR-associated endonuclease Cpf1 to site-specifically cleave a target DNA, e.g., protospacer sequence.

In one embodiment, one or more crRNAs or sgRNAs contemplated herein, can be designed to target a gene associated with a disease or condition set forth in Table 2 or Table 3.

In one embodiment, one or more crRNAs or sgRNAs contemplated herein, can be designed to target a gene associated with a channelopathy.

In one embodiment, one or more crRNAs or sgRNAs contemplated herein, can be designed to target nociceptive genes and genes associated with the regulation of pain. In various embodiments, the one or more crRNAs or sgRNAs contemplated herein, can be designed to target a voltage gated ion channel, such as a voltage gated sodium or potassium channel.

Illustrative examples of voltage gated sodium channel that are suitable for targeting with crRNAs or sgRNAs contemplated herein include, but are not limited to: Nav1.1, Nav1.2, Nav1.3, Nav1.5, Nav1.6, Nav1.7, Nav1.8, and Nav1.9.

In one embodiment, the one or more crRNAs comprises a pair of offset crRNAs complementary to opposite strands of the target site. In one embodiment, the one or more sgRNAs comprises a pair of offset sgRNAs complementary to opposite strands of the target site. Without wishing to be bound by any particular theory, in some embodiments, it is contemplated that using a pair of offset crRNAs or sgRNAs with a Cas9 or Cpf1 nickase contemplated herein reduces off target genome editing. A single nick is repaired efficiently using a cell's base-excision-repair (BER) machinery. Thus, a large majority of single nicks do not result in nonhomologous end joining (NHEJ)-mediated indels. By inducing offset nicks, off-target single nick events will likely result in very low indel rates.

In one embodiment, offset nicks are induced using a pair of offset crRNAs or sgRNAs with a Cas9 or Cpf1 nickase increases site-specific NHEJ or HDR (when a repair template is provided). In one embodiment, a pair of offset crRNAs or sgRNAs is designed to create 5′ overhangs via the offset nicks to increase the rate of site-specific NHEJ or homologous recombination.

In one embodiment, the pair of offset crRNAs or sgRNAs are offset by at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 88, 89, 90, 91, 92, 93, 94, 95, 96, 97, 98, 99, or at least 100 nucleotides.

In one embodiment, the pair of offset crRNAs or sgRNAs are offset by about 5 to about 100 nucleotides, about 10 to about 50 nucleotides, about 10 to about 40 nucleotides, about 10 to about 30 nucleotides, about 10 to about 20 nucleotides, or about 15 to 30 nucleotides, as well as all intermediate lengths or ranges.

In one embodiment, a crRNA or sgRNA is designed to induce a single nick with a Cas9 or Cpf1 nickase; in combination with a double-stranded or single-stranded repair template polynucleotide, the nick is repaired using homologous recombination with minimal off-target indel effects.

Illustrative examples for bacterial sources of Cas9 polynucleotides encoding a Cas9 polypeptide suitable for use in the methods contemplated herein and corresponding PAM motifs include, but are not limited to: Staphylococcus aureus, (NNGRR), Streptococcus pyogenes Cas9 (NGG); Streptococcus thermophilis Cas9 (NNNNGANN, NNNNGTTN, NNNNGNNT, NNAGAAW, NNNNGTNN, NNNNGNTN); Treponema denticola Cas9 (NAAAAN, NAAANC, NANAAC, NNAAAC); and Neisseria meningitidis Cas9 (NNAGAA, NNAGGA, NNGGAA, NNNNGATT, NNANAA, NNGGGA). Without wishing to be bound to any particular theory, a virtually limitless selection of protospacer motifs may be targeted using the CRISPR technology because a suitable Cas9 may be selected to target any protospacer based on the sequence of the adjacent PAM motif.

Illustrative examples for bacterial sources of Cpf1 polynucleotides encoding a Cpf1 polypeptide suitable for use in the methods contemplated herein include, but are not limited to: Francisella novicida, Acidaminococcus sp. BV3L6, and Lachnospiraceae bacterium ND2006.

As used herein, the terms “siRNA” or “short interfering RNA” refer to a short polynucleotide sequence that mediates a process of sequence-specific post-transcriptional gene silencing, translational inhibition, transcriptional inhibition, or epigenetic RNAi in animals (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886). In certain embodiments, an siRNA comprises a first strand and a second strand that have the same number of nucleosides; however, the first and second strands are offset such that the two terminal nucleosides on the first and second strands are not paired with a residue on the complimentary strand. In certain instances, the two nucleosides that are not paired are thymidine resides. The siRNA should include a region of sufficient homology to the target gene, and be of sufficient length in terms of nucleotides, such that the siRNA, or a fragment thereof, can mediate down regulation of the target gene. Thus, an siRNA includes a region which is at least partially complementary to the target RNA. It is not necessary that there be perfect complementarity between the siRNA and the target, but the correspondence must be sufficient to enable the siRNA, or a cleavage product thereof, to direct sequence specific silencing, such as by RNAi cleavage of the target RNA. Complementarity, or degree of homology with the target strand, is most critical in the antisense strand. While perfect complementarity, particularly in the antisense strand, is often desired, some embodiments include one or more, but preferably 10, 8, 6, 5, 4, 3, 2, or fewer mismatches with respect to the target RNA. The mismatches are most tolerated in the terminal regions, and if present are preferably in a terminal region or regions, e.g., within 6, 5, 4, or 3 nucleotides of the 5′ and/or 3′ terminus. The sense strand need only be sufficiently complementary with the antisense strand to maintain the overall double-strand character of the molecule. Each strand of an siRNA can be equal to or less than 30, 25, 24, 23, 22, 21, or 20 nucleotides in length. The strand is preferably at least 19 nucleotides in length. For example, each strand can be between 21 and 25 nucleotides in length. Preferred siRNAs have a duplex region of 17, 18, 19, 29, 21, 22, 23, 24, or 25 nucleotide pairs, and one or more overhangs of 2-3 nucleotides, preferably one or two 3′ overhangs, of 2-3 nucleotides.

As used herein, the terms “miRNA” or “microRNA” s refer to small non-coding RNAs of 20-22 nucleotides, typically excised from ˜70 nucleotide foldback RNA precursor structures known as pre-miRNAs. miRNAs negatively regulate their targets in one of two ways depending on the degree of complementarity between the miRNA and the target. First, miRNAs that bind with perfect or nearly perfect complementarity to protein-coding mRNA sequences induce the RNA-mediated interference (RNAi) pathway. miRNAs that exert their regulatory effects by binding to imperfect complementary sites within the 3′ untranslated regions (UTRs) of their mRNA targets, repress target-gene expression post-transcriptionally, apparently at the level of translation, through a RISC complex that is similar to, or possibly identical with, the one that is used for the RNAi pathway. Consistent with translational control, miRNAs that use this mechanism reduce the protein levels of their target genes, but the mRNA levels of these genes are only minimally affected. miRNAs encompass both naturally occurring miRNAs as well as artificially designed miRNAs that can specifically target any mRNA sequence. For example, in one embodiment, the skilled artisan can design short hairpin RNA constructs expressed as human miRNA (e.g., miR-30 or miR-21) primary transcripts or “mishRNA.” This design adds a Drosha processing site to the hairpin construct and has been shown to greatly increase knockdown efficiency (Pusch et al., 2004). The hairpin stem consists of 22-nt of dsRNA (e.g., antisense has perfect complementarity to desired target) and a 15-19-nt loop from a human miR. Adding the miR loop and miR30 flanking sequences on either or both sides of the hairpin results in greater than 10-fold increase in Drosha and Dicer processing of the expressed hairpins when compared with conventional shRNA designs without microRNA. Increased Drosha and Dicer processing translates into greater siRNA/miRNA production and greater potency for expressed hairpins.

In one embodiment, a polynucleotide encoding a CRISPR-Cas endonuclease comprises an intron that comprises a miRNA and a 3′UTR that comprises a corresponding miRNA target site. Without wishing to be bound to any particular theory, it is contemplated that this architecture can be used to transiently regulate the expression of the CRISPR-Cas endonuclease and minimize the off-target effects of the endonuclease either alone or in combination with one or more additional regulatory elements to regulate the transient expression of the endonuclease.

As used herein, the terms “shRNA” or “short hairpin RNA” refer to double-stranded structure that is formed by a single self-complementary RNA strand. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Greater than 90% sequence identity, or even 100% sequence identity, between the inhibitory RNA and the portion of the target gene is preferred. In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

As used herein, the term “ribozyme” refers to a catalytically active RNA molecule capable of site-specific cleavage of target mRNA. Several subtypes have been described, e.g., hammerhead and hairpin ribozymes. Ribozyme catalytic activity and stability can be improved by substituting deoxyribonucleotides for ribonucleotides at noncatalytic bases. While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction and production of hammerhead ribozymes is well known in the art.

In one embodiment, an expression cassette comprises one or more of a crRNA, a tracrRNA, sgRNA, an siRNA, an miRNA, an shRNA, or a ribozyme and further comprises one or more regulatory sequences, such as, for example, a strong constitutive RNA pol III promoter, e.g., human or mouse U6 snRNA promoter, the human and mouse H1 RNA promoter, or the human tRNA-val promoter; an inducible RNA pol III promoter, e.g., U6-6TetO promoter, H1-peroxide promoter; or a strong constitutive or inducible RNA pol II promoter, as described elsewhere herein.

The polynucleotides contemplated herein, regardless of the length of the coding sequence itself, may be combined with other DNA sequences, such as expression control sequences, regulatory elements, promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), guide RNA target sites, termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated and/or expressed using any of a variety of well established techniques known and available in the art. In order to express a desired polypeptide, a nucleotide sequence encoding the polypeptide, can be inserted into an appropriate vector, such as a viral vector.

Illustrative examples of viral vectors suitable for use in particular embodiments include, but are not limited to lentiviral vectors, adenovirus vectors, and adeno-associated virus (AAV) vectors. In preferred embodiments, the viral vector is an AAV vector.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide for use in practicing the invention is a vector, including but not limited to expression vectors and viral vectors, and includes exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide. In particular embodiments, the vector comprises one or more RNA pol II and/or RNA pol III promoters.

Illustrative examples of RNA pol II promoters suitable for use in particular embodiments include, but are not limited to a neuron specific promoter.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) or regulatory element and a second polynucleotide sequence, e.g., a polynucleotide-of-interest, wherein the expression control sequence or regulatory element directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments of the invention include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

In a particular embodiments, it may be desirable to use a tissue-specific promoter to achieve cell-type specific, lineage specific, or tissue-specific expression of a desired polynucleotide sequence. Any of a wide variety of tissue-specific promoters are known to those skilled in the art with respect to cell and tissue types of interest. For example, in certain embodiments, illustrative tissue-specific promoters include, but are not limited to: a glial fibrillary acidic protein (GFAP) promoter (astrocyte expression), a synapsin promoter (neuron expression), and calcium/calmodulin-dependent protein kinase II (neuron expression), tubulin alpha I (neuron expression), neuron-specific enolase (neuron expression), platelet-derived growth factor beta chain (neuron expression), a TRPV1 promoter (neuron expression), a Nav1.7 promoter (neuron expression), a Nav1.8 promoter (neuron expression), a Nav1.9 promoter (neuron expression), or an Advillin promoter (neuron expression).

According to certain embodiments, the cell type specific promoter is specific for cell types found in the brain (e.g., neurons, glial cells).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments of the invention provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Illustrative examples of promoters suitable for use in particular embodiments include, but are not limited to neuron specific promoters.

In particular embodiments, a polynucleotide contemplated herein comprises a neuron specific promoter or a promoter operative in a neuronal cell.

In particular embodiments, a polynucleotide contemplated herein comprises a neuron specific promoter operable in a trigeminal ganglion (TGG) neuron or a dorsal root ganglion (DRG) neuron.

In particular embodiments, a polynucleotide contemplated herein comprises a neuron specific promoter selected from the group consisting of a calcium/calmodulin-dependent protein kinase II promoter, a tubulin alpha I promoter, a neuron-specific enolase promoter, a platelet-derived growth factor beta chain promoter, an hSYN1 promoter, a TRPV1 promoter, a Nav1.7 promoter, a Nav1.8 promoter, a Nav1.9 promoter, and an Advillin promoter.

In one embodiment, the neuron specific promoter is a human synapsin 1 (SYN1) promoter.

In particular embodiments, polynucleotides contemplated herein comprise at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments of the present invention include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. As used herein, the terms “recombination sequence,” “recombination site,” or “site specific recombination site” refer to a particular nucleic acid sequence to which a recombinase recognizes and binds.

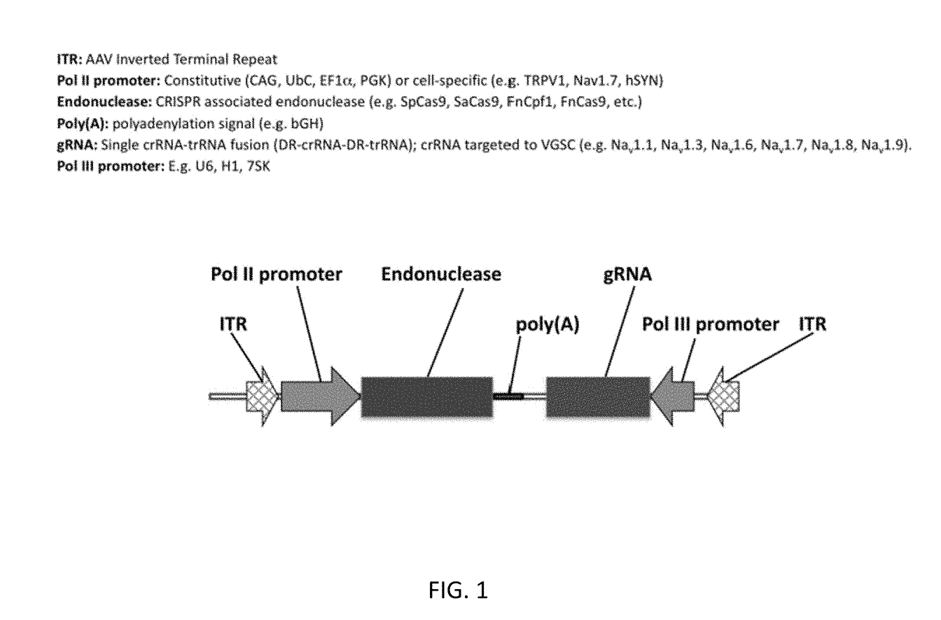

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

In particular embodiments, polynucleotides contemplated herein, include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, a polynucleotide encoding a polypeptide comprises a consensus Kozak sequence. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:56), where R is a purine (A or G) (Kozak, 1986. Cell. 44(2):283-92, and Kozak, 1987. Nucleic Acids Res. 15(20):8125-48).

In particular embodiments, polynucleotides comprise a polyadenylation sequence 3′ of a polynucleotide encoding a polypeptide to be expressed. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments the poly(A) sequence is an SV40 polyA sequence, a bovine growth hormone polyA sequence (BGHpA), a rabbit β-globin polyA sequence (rβgpA), or another suitable heterologous or endogenous polyA sequence known in the art.

### D. Polypeptides

The present invention contemplates, in part, compositions comprising polypeptides, e.g., switch polypeptides, CRISPR-Cas endonucleases; fusion polypeptides; and vectors that express polypeptides.

“Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

An “isolated peptide” or an “isolated polypeptide” and the like, as used herein, refer to in vitro isolation, purification, recombinant production, or synthesis of a peptide or polypeptide molecule from a cellular environment, and from association with other components of the cell, i.e., it is not significantly associated with in vivo substances.

A “swtich polypeptde” refers to a polypeptide that binds an inducible regulatory element or regulatory element for inducible expression contemplated herein. Illustrative examples of switch polypeptides suitable for use in particular embodiments include, but are not limited to, a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

Polypeptides include biologically active “polypeptide fragments.” As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. Polypeptide fragments refer to a polypeptide, which can be monomeric or multimeric, that has an amino-terminal deletion, a carboxyl-terminal deletion, and/or an internal deletion or substitution of one or more amino acids of a naturally-occurring or recombinantly-produced polypeptide. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to improve the biological properties of a polypeptide or the binding or cleavage specificity of a Cas or Cpf1 polypeptide by introducing one or more substitutions, deletions, additions and/or insertions into the polypeptide. Preferably, polypeptides of the invention include polypeptides having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity thereto.

As noted above, polypeptides contemplated herein may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides of the present invention and still obtain a functional molecule that encodes a variant or derivative polypeptide with desirable characteristics. When it is desired to alter the amino acid sequence of a polypeptide to create an equivalent, or even an improved, variant polypeptide of the invention, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |
| Threonine | T | Thr | ACA | ACC | ACG | ACU | | |
| Valine | V | Val | GUA | GUC | GUG | GUU | | |
| Tryptophan | W | Trp | UGG | | | | | |
| Tyrosine | Y | Tyr | UAC | UAU | | | | |

Guidance in determining which amino acid residues can be substituted, inserted, or deleted without abolishing biological activity can be found using computer programs well known in the art, such as DNASTAR′ software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In various embodiments, Cas9 polypeptides are contemplated. Cas9 is the signature protein characteristic for type II CRISPR nuclease systems in bacteria. At least 235 Cas9 orthologs have been identified in 203 bacterial species, the names and sequences of which are herein incorporated by reference in their entirety from the publication and supplemental information of Chylinski et al., 2013. RNA Biol. 10(5): 726-737. Conserved regions of Cas9 orthologs include a central HNH endonuclease domain and a split RuvC/RNase H domain.

In particular embodiments, a suitable Cas9 polypeptide sequence may be obtained from the following illustrative list of bacterial species: Staphylococcus aureus, Enterococcus faecium, Enterococcus italicus, Listeria innocua, Listeria monocytogenes, Listeria seeligeri, Listeria ivanovii, Streptococcus agalactiae, Streptococcus anginosus, Streptococcus bovis, Streptococcus dysgalactiae, Streptococcus equinus, Streptococcus gallolyticus, Streptococcus macacae, Streptococcus mutans, Streptococcus pseudoporcinus, Streptococcus pyogenes, Streptococcus thermophilus, Streptococcus gordonii, Streptococcus infantarius, Streptococcus macedonicus, Streptococcus mitis, Streptococcus pasteurianus, Streptococcus suis, Streptococcus vestibularis, Streptococcus sanguinis, Streptococcus downei, Neisseria bacilliformis, Neisseria cinerea, Neisseria flavescens, Neisseria lactamica, Neisseria meningitidis, Neisseria subflava, Lactobacillus brevis, Lactobacillus buchneri, Lactobacillus casei, Lactobacillus paracasei, Lactobacillus fermentum, Lactobacillus gasseri, Lactobacillus jensenii, Lactobacillus johnsonii, Lactobacillus rhamnosus, Lactobacillus ruminis, Lactobacillus salivarius, Lactobacillus sanfranciscensis, Corynebacterium accolens, Corynebacterium diphtheriae, Corynebacterium matruchotii, Campylobacter jejuni, Clostridium perfringens, Treponema vincentii, Treponema phagedenis, and Treponema denticola.

Cas9 polypeptides target double-stranded polynucleotide sequences flanked by particular 3′ PAM sequences specific to a particular Cas9 polypeptide. Each Cas9 nuclease domain cleaves one DNA strand. Cas9 polypeptides naturally contain domains homologous to both HNH and RuvC endonucleases. The HNH and RuvC-like domains are each responsible for cleaving one strand of the double-stranded DNA target sequence. The HNH domain of the Cas9 polypeptide cleaves the DNA strand complementary to the tracrRNA:crRNA or sgRNA. The RuvC-like domain of the Cas9 polypeptide cleaves the DNA strand that is not-complementary to the tracrRNA:crRNA or sgRNA.

In one embodiment, a Cas9 polypeptide or biologically active fragment thereof comprising catalytic activity of the HNH and RuvC domains is contemplated.

In particular embodiments, a Cas9 polypeptide variant is contemplated comprising one or more amino acids additions, deletions, mutations, or substitutions in the HNH or RuvC-like endonuclease domains that decreases or eliminates the nuclease activity of the variant domain. In one embodiment, the variant is a Cas9 nickase.

In one embodiment, the Cas9 polypeptide is catalytically inactive, meaning that one or more amino acids additions, deletions, mutations, or substitutions in the HNH and the RuvC-like endonuclease domains have been made to render the Cas9 catalytically inactive.

In one embodiment, a Cas9 polypeptide comprises one or more amino acids additions, deletions, mutations, or substitutions that decrease or eliminate the nuclease activity in the HNH domain. Illustrative examples of Cas9 enzymes and corresponding mutations that decrease or eliminate the nuclease activity in the HNH domain include, but are not limited to: S. aureus (D10A), S. pyogenes (D10A); S. thermophilis (D9A); T. denticola (D13A); and N. meningitidis (D16A).

In one embodiment, a Cas9 polypeptide comprises one or more amino acids additions, deletions, mutations, or substitutions that decrease or eliminate the nuclease activity in the RuvC-like domain. Illustrative examples of Cas9 enzymes and corresponding mutations that decrease or eliminate the nuclease activity in the RuvC-like domain include, but are not limited to: S. aureus (N580A), S. pyogenes (D839A, H840A, or N863A); S. thermophilis (D598A, H599A, or N622A); T. denticola (D878A, H879A, or N902A); and N. meningitidis (D587A, H588A, or N611A).

In one embodiment, a Cas9 nickase and one or more guide RNAs comprising a pair of offset guide RNAs complementary to opposite strands of the target site are used to engineer a double-strand break. In one embodiment, a pair of offset guide RNAs is designed to create 5′ overhangs via the offset nicks to increase the rate of site-specific NHEJ or homologous recombination when a DNA repair template is present.

In one embodiment, a Cas9 nickase and a guide RNA designed against a target sequence is used to engineer a single-strand break. In one embodiment, a guide RNA in combination with a Cas9 nickase and a double-stranded or single-stranded repair template is used to engineer a single-strand break that is repaired using homologous recombination with minimal off-target effects.

In various embodiments, Cpf1 polypeptides are contemplated.

In particular embodiments, a suitable Cpf1 polypeptide sequence may be obtained from the following illustrative list of bacterial species: Francisella novicida, Acidaminococcus sp. BV3L6, or Lachnospiraceae bacterium ND2006.

In one embodiment, a Cpf1 polypeptide comprises one or more amino acids additions, deletions, mutations, or substitutions that decrease or eliminate the nuclease activity in the RuvC-like domain.

Illustrative examples of Cpf1 enzymes and corresponding mutations that decrease or eliminate the nuclease activity in the RuvC-like domain include, but are not limited to: Cpf1 from Francisella novicida, wherein the mutation is a D917A, E1006A, or D1225A mutation.

Polypeptides of the present invention include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

Fusion polypeptides can comprise one or more polypeptide domains or segments including, but are not limited to cell permeable peptide domains (CPP), Zn-finger DNA binding domains, nuclease domains, chromatin remodeling domains, histone modifying domains, and epigenetic modifying domains, epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, and interspecies homologs, so long as the desired transcriptional activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as discussed elsewhere herein.

In various embodiments, a fusion polypeptide comprising a Cas9 or Cpf1 endonuclease, nickase, or catalytically inactive mutant is contemplated. In one embodiment, a fusion polypeptide comprises a catalytically inactive Cas9 or Cpf1 polypeptide and a nuclease domain. In particular embodiments, the fusion polypeptide comprises an endonuclease domain that is a cleavage half-domain, such as, for example, the cleavage domain of a Type IIs restriction endonuclease such as FokI. A pair of such nuclease half-domain fusions is used for targeted cleavage for each strand of the target

In various embodiments, the fusion polypeptide or a switch fusion polypeptide comprises one or more functional domains selected from the group consisting of: a histone methylase or demethylase domains, a histone acetylase or deacetylase domains, a SUMOylation domain, an ubiquitylation or deubiquitylation domain, a DNA methylase or DNA demethylase domain, and a nuclease domain.

In one embodiment, the nuclease domain is a FOK I cleavage domain.

In one embodiment, the nuclease domain is a TREX2 domain.

Fusion polypeptides may optionally comprise a linker that can be used to link the one or more polypeptides. A peptide linker sequence may be employed to separate any two or more polypeptide components by a distance sufficient to ensure that each polypeptide folds into its appropriate secondary and tertiary structures so as to allow the polypeptide domains to exert their desired functions. Such a peptide linker sequence is incorporated into the fusion polypeptide using standard techniques in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. Linker sequences are not required when a particular fusion polypeptide segment contains non-essential N-terminal amino acid regions that can be used to separate the functional domains and prevent steric interference. Preferred linkers are typically flexible amino acid subsequences which are synthesized as part of a recombinant fusion protein. Linker polypeptides can be between 1 and 200 amino acids in length, between 1 and 100 amino acids in length, or between 1 and 50 amino acids in length, including all integer values in between.

Exemplary linkers include, but are not limited to the following amino acid sequences: DGGGS (SEQ ID NO:57); TGEKP (SEQ ID NO:58) (see, e.g., Liu et al., PNAS 5525-5530 (1997)); GGRR (SEQ ID NO:59) (Pomerantz et al. 1995, supra); (GGGGS)ₙ (SEQ ID NO:60) (Kim et al., PNAS 93, 1156-1160 (1996.); EGKSSGSGSESKVD (SEQ ID NO:61) (Chaudhary et al., 1990, Proc. Natl. Acad. Sci. U.S.A. 87:1066-1070); KESGSVSSEQLAQFRSLD (SEQ ID NO:62) (Bird et al., 1988, Science 242:423-426), GGRRGGGS (SEQ ID NO:63); LRQRDGERP (SEQ ID NO:64); LRQKDGGGSERP (SEQ ID NO:65); LRQKd(GGGS)₂ERP (SEQ ID NO:66). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods.

Fusion polypeptides may further comprise a polypeptide cleavage signal between each of the polypeptide domains described herein. In addition, polypeptide site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO:67), for example, ENLYFQG (SEQ ID NO:68) and ENLYFQS (SEQ ID NO:69), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide. In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

### E. Viral Vectors

In various embodiments, a vector comprises a one or more polynucleotide sequences contemplated herein. The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Illustrative examples of suitable vectors include, for example, plasmids (e.g., DNA plasmids or RNA plasmids), transposons, cosmids, bacterial artificial chromosomes, and viral vectors.

Illustrative examples of viral vectors suitable for use in delivering polynucleotides contemplated herein include, but are not limited to adeno-associated virus, retrovirus, lentivirus, and adenovirus.

In various embodiments, the vectors contemplated herein have been altered to render them suitable for delivering a genome editing platform to a desired cell or tissue type. For example, in certain specific embodiments, the vectors are suitable for delivering a genome editing platform to an excitable cell in order to treat, prevent, ameliorate, or manage pain. Without wishing to be bound by any particular theory, the gene therapy vectors contemplated herein provide numerous advantages over existing vectors because they are engineered to reduce off-target genome editing, because they are engineered to express a plurality of expression cassettes necessary to reconstitute an entire gene editing platform in a single vector; and because they are engineered for efficient delivery in vitro, in vivo, or ex vivo to cells of interest, such as excitable cells involved in the regulation of pain.

As will be evident to one of skill in the art, the term “viral vector” is widely used to refer either to a nucleic acid molecule that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s).

In various embodiments, a vector contemplated herein comprises a polynucleotide that is an inducibly and transiently regulatable gene editing system. The vectors comprise one or more expression cassettes that together constitute a genome editing platform for gene therapy. In a preferred embodiment, the genome editing system is a CRISPR-Cas endonuclease gene editing system. The components of the CRISPR-Cas system may be inserted into one or more expression cassettes which are in turn engineered into the vector.

In preferred embodiments, the vectors contemplated herein provide the advantage of delivering a complete genome editing platform in a single vector and also provide a more efficient genome editing platform that is less prone to off-target effects. Without wishing to be bound to a particular theory, the vectors contemplated herein provide inducibly and/or transiently regulatable CRISPR-Cas endonucleases (e.g., Cas9, Cpf1) to reduce off-target effects. In particular embodiments, the CRISPR-Cas endonuclease is transiently expressed on the order of minute, hours, or days. In particular embodiments, the CRISPR-Cas endonuclease is transiently expressed for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes or more. In certain embodiments, the CRISPR-Cas endonuclease is transiently expressed for about 1, 2, 3, 4, 5, 6, 7, 8, 9, 10, 11, or 12 hours.

In particular embodiments, a vector comprises a genome editing platform for knocking out genes or altering the activity of cis-acting genetic regulatory elements in the genome.

In particular embodiments, a vector comprises a genome editing platform for making corrections to the genome or inserting genetic material into the genome.

In various embodiments, a vector comprises one or a plurality of expression cassettes encoding a transiently and inducibly regulatable CRISPR-Cas endonuclease, a polynucleotide encoding one or more guide RNAs, a polynucleotide encoding a switch polypeptide that induces expression of the CRISPR-Cas endonuclease and/or the guide RNAs, and optionally a DNA template for altering the genome. The vector may be transiently regulated by flanking the CRISPR-Cas endonuclease and/or the switch polypeptide with guide RNA target sites that match the genome target sites, thereby inactivating the vector and accomplishing the desired genome editing strategy. Thus, the genome editing platform and related vectors contemplated herein provide a quantum leap in genome editing safety compared to existing strategies.

In one embodiment, a vector comprises a nucleic acid comprising an inducibly and transiently regulatable CRISPR-Cas system for the treatment, prevention, or amelioration of a disease or condition disclosed herein.

In one embodiment, a vector comprises a first expression cassette that comprises an RNA polymerase II promoter operably linked to a polynucleotide encoding a CRISPR-Cas endonuclease; and a second expression cassette that comprises one or more RNA polymerase III promoters each operably linked to a polynucleotide encoding one or more guide RNAs.

In another embodiment, a vector comprises a first expression cassette that comprises at least one regulatory element for transient expression and an RNA polymerase II promoter operably linked to a polynucleotide encoding a CRISPR-Cas endonuclease; and a second expression cassette that comprises one or more RNA polymerase III promoters each operably linked to a polynucleotide encoding one or more guide RNAs.

In yet another embodiment, a vector comprises a first expression cassette that comprises at least one regulatory element for inducible expression and at least one regulatory element for transient expression and a polynucleotide encoding a CRISPR-Cas endonuclease; a second expression cassette that comprises one or more RNA polymerase III promoters each operably linked to a polynucleotide encoding one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In yet another embodiment, a vector comprises a first expression cassette that comprises at least one regulatory element for transient expression and an RNA polymerase II promoter operably linked to a polynucleotide encoding a CRISPR-Cas endonuclease; a second expression cassette that comprises at least one regulatory element for inducible expression operably linked to a polynucleotide encoding one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In still yet another embodiment, a vector comprises a first expression cassette that comprises at least one regulatory element for inducible expression and at least one regulatory element for transient expression and a polynucleotide encoding a CRISPR-Cas endonuclease; a second expression cassette that comprises at least two RNA polymerase III promoters each operably linked to one or more guide RNAs; and a third expression cassette that comprises at least one regulatory element for transient expression and an RNA polymerase II promoter operably linked to a polynucleotide encoding a switch polypeptide, wherein the switch polypeptide binds to the at least one element for inducible expression.

In a particular embodiment, one or more crRNAs or sgRNAs contemplated herein, can be designed to target guide RNA target sites in the vector as well as genes associated diseases described herein, such as genes associated with diseases set forth in Table 2 or Table 3, or nociceptive genes or genes involved in the regulation of pain.

In one embodiment, the vector has a polynucleotide encoding the CRISPR-Cas endonuclease that also encodes an inhibitory RNA and contains a recognition site for the inhibitory RNA, which provides the vector with yet another layer of control of CRISPR-Cas endonuclease expression.

In particular embodiments, the Cas9, Cpf1, and/or switch polypeptides are fusion polypeptides, optionally fused to a nuclease domain, including, without limitation, a FOK I nuclease domain or a TREX2 domain.

In other particular embodiments, the Cas9, Cpf1, and/or switch polypeptide is a polypeptide comprising a self-cleaving viral peptide and TREX2.

One of the major advantages of the vectors contemplated herein, is the ability to transiently regulate the activity of the genome editing platform by flanking the expression cassettes or the polynucleotides therein and/or a DNA donor template for altering the genome by one or more guide RNAs, e.g., crRNAs or sgRNAs. When crRNAs are used a corresponding tracRNA is required for each target site for Cas9; tracRNA is not required for cleavage with Cpf1. In one embodiment, the one or more guide RNAs recognize the guide RNA target sites flanking the expression cassettes to inactivate (by excision) the desired components of the genome editing platform and optionally to release the DNA template. In one embodiment, the guide RNA target site flanking the 5′ end of a polynucleotide to be deleted and the guide RNA target site flanking the 3 ‘of the polynucleotide to be inactivated and optionally to release the DNA template are the same. In one embodiment, the 5’ and 3′ guide RNA target sites flanking the polynucleotide to be inactivated and optionally to release the DNA template are different. In one embodiment, the 5′ and 3′ guide RNA target sites flanking the polynucleotide to be inactivated are the same for all flanked nucleotides and optionally to release the DNA template. In one embodiment, the 5′ and 3′ guide RNA target sites flanking the polynucleotide to be inactivated are different for all flanked nucleotides and optionally to release the DNA template, but in some embodiments, all the 5′ guide RNA target sites are the same and all the 3′ guide RNA target sites are the same.

In various embodiments, the same one or more guide RNAs target both the guide RNA targets sites in the vector as well as the target sequence in the genome. See, e.g., SEQ ID NOs: 1-55.

In particular embodiments, vectors comprises one or more expression cassettes comprising a RNA pol II promoter. The promoters may be ubiquitous or constitutive RNA pol II promoters, tissue or lineage-specific RNA pol II promoters, or inducible RNA pol II promoters. In a vector comprising multiple RNA pol II promoters, the promoters may be the same or different.

Illustrative examples of ubiquitous RNA pol II promoters useful in certain embodiments contemplated herein include, but are not limited to cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

Illustrative examples of tissue specific or lineage specific RNA pol II promoters useful in certain embodiments contemplated herein include, but are not limited to a neuron specific promoter, a promoter operable in a trigeminal ganglion (TGG) neuron, a dorsal root ganglion (DRG) neuron, an hSYN1 promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter.

In one embodiment, the tissue specific or lineage specific RNA pol II promoter is selected from the group consisting of hSYN1 promoter, a TRPV1 promoter, a Nav1.7 promoter, a Nav1.8 promoter, and a Nav1.9 promoter.

In particular embodiments, a vector comprises a switch polypeptide selected from the group consisting of a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

Illustrative examples of regulatory elements for inducible expression include, but are not limited to a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

In particular embodiments, a vector comprises one or more expression cassettes comprising a RNA pol III promoter. The promoters may be ubiquitous or constitutive RNA pol III promoters or inducible RNA pol III promoters.

Illustrative examples of ubiquitous RNA pol III promoters useful in certain embodiments contemplated herein include, but are not limited to a human U6 snRNA promoter, a mouse U6 snRNA promoter, a human H1 RNA promoter, a mouse H1 RNA promoter, and a human tRNA-val promoter.

Illustrative examples of inducible RNA pol III promoters useful in certain embodiments contemplated herein include, but are not limited to an RNA pol III promoter operably linked to a tetracycline responsive regulatory element or a peroxide inducible regulatory element.

In particular embodiments, the vector comprises a polynucleotide encoding a CRISPR-Cas endonuclease selected from the group consisting of: Cpf1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4. In a preferred embodiment, the Cas is Cas9 or Cpf1. In particular embodiments, the Cas9 or Cpf1 may comprise one or more mutations in a HNH or a RuvC-like endonuclease domain or the HNH and the RuvC-like endonuclease domains as disclosed elsewhere herein.

In particular embodiments, a vector contemplated herein comprises a polynucleotide encoding a DNA template for altering at least one site in a genome. The alteration may comprise correction of one or more genome sequences or insertion of sequences into the genome.

In some embodiments, the editing of the genome in the cell comprises insertion of a regulatable transcriptional regulatory element upstream of a transcription start site in a gene of the cell.

In certain embodiments, the transcriptional regulatory element may be activated in the presence of an exogenous ligand or small molecule or activated in the absence of an exogenous ligand or small molecule.

In certain other embodiments, the transcriptional regulatory element may be repressed in the presence of an exogenous ligand or small molecule or repressed in the absence of an exogenous ligand or small molecule.

In one embodiment, the transcriptional regulatory element is inserted upstream of a gene associated with a disease set forth in Table 2 or Table 3.

In one embodiment, the transcriptional regulatory element is inserted upstream of a gene associated with a channelopathy, particularly a human channelopathy.

In one embodiment, the transcriptional regulatory element is inserted upstream of a gene associated with the regulation of pain.

In a particular embodiment, the transcriptional regulatory element is inserted upstream of a gene encoding a voltage gated ion channel, such as a voltage gated sodium or potassium channel.

Illustrative examples of voltage gated sodium channels include, but are not limited to: Naᵥ1.1, Naᵥ1.2, Naᵥ1.3, Naᵥ1.5, Naᵥ1.6, Naᵥ1.7, Naᵥ1.8, and Naᵥ1.9.

1. Adeno-Associated Virus (AAV) Vectors

In one embodiment, the vector is a viral vector. Illustrative examples of suitable viral vectors include, but are not limited to, retroviral vectors (e.g., lentiviral vectors), herpes virus based vectors and parvovirus based vectors (e.g., adeno-associated virus (AAV) based vectors, AAV-adenoviral chimeric vectors, and adenovirus-based vectors).

The term “parvovirus” as used herein encompasses all parvoviruses, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, mouse minute virus, bovine parvovirus, canine parvovirus, chicken parvovirus, feline panleukopenia virus, feline parvovirus, goose parvovirus, and B19 virus. Other autonomous parvoviruses are known to those skilled in the art. See, e.g., Fields et al., 1996 Virology, volume 2, chapter 69 (3d ed., Lippincott-Raven Publishers).

The genus Dependovirus contains the adeno-associated viruses (AAV), including but not limited to, AAV type 1, AAV type 2, AAV type 3, AAV type 4, AAV type 5, AAV type 6, avian AAV, bovine AAV, canine AAV, equine AAV, and ovine AAV.

In a preferred embodiment, the vector is an AAV vector.

The genomic organization of all known AAV serotypes is similar. The genome of AAV is a linear, single-stranded DNA molecule that is less than about 5,000 nucleotides (nt) in length. Inverted terminal repeats (ITRs) flank the unique coding nucleotide sequences for the non-structural replication (Rep) proteins and the structural (VP) proteins. The VP proteins (VP1,-2 and -3) form the capsid and contribute to the tropism of the virus. The terminal 145 nt ITRs are self-complementary and are organized so that an energetically stable intramolecular duplex forming a T-shaped hairpin may be formed. These hairpin structures function as an origin for viral DNA replication, serving as primers for the cellular DNA polymerase complex. Following wild-type (wt) AAV infection in mammalian cells the Rep genes are expressed and function in the replication of the viral genome.

A “recombinant parvoviral or AAV vector” (or “rAAV vector”) herein refers to a vector comprising one or more polynucleotides contemplated herein that are flanked by one or more AAV ITRs. Such rAAV vectors can be replicated and packaged into infectious viral particles when present in an insect host cell that is expressing AAV rep and cap gene products (i.e., AAV Rep and Cap proteins). When an rAAV vector is incorporated into a larger nucleic acid construct (e.g., in a chromosome or in another vector such as a plasmid or baculovirus used for cloning or transfection), then the rAAV vector is typically referred to as a “pro-vector” which can be “rescued” by replication and encapsidation in the presence of AAV packaging functions and necessary helper functions.

In particular embodiments, any AAV ITR may be used in the AAV vectors, including ITRs from AAV1, AAV2, AAV3, AAV4, AAV5, AAV6, AAV7, AAV8, AAV9, AAV10, AAV11, AAV12, AAV13, AAV14, AAV15, and AAV16. In one preferred embodiment, an AAV vector contemplated herein comprises one or more AAV2 ITRs.

rAAV vectors comprising two ITRs have a payload capacity of about 4.4 kB.

Self-complementary rAAV vectors contain a third ITR and package two strands of the recombinant portion of the vector leaving only about 2.1 kB for the polynucleotides contemplated herein. In one embodiment, the AAV vector is an scAAV vector.

Extended packaging capacities that are roughly double the packaging capacity of an rAAV (about 9 kB) have been achieved using dual rAAV vector strategies. Dual vector strategies useful in producing rAAV contemplated herein include, but are not limited to splicing (trans-splicing), homologous recombination (overlapping), or a combination of the two (hybrid). In the dual AAV trans-splicing strategy, a splice donor (SD) signal is placed at the 3′ end of the 5′-half vector and a splice acceptor (SA) signal is placed at the 5′ end of the 3′-half vector. Upon co-infection of the same cell by the dual AAV vectors and inverted terminal repeat (ITR)-mediated head-to-tail concatemerization of the two halves, trans-splicing results in the production of a mature mRNA and full-size protein (Yan et al., 2000). Trans-splicing has been successfully used to express large genes in muscle and retina (Reich et al., 2003; Lai et al., 2005). Alternatively, the two halves of a large transgene expression cassette contained in dual AAV vectors may contain homologous overlapping sequences (at the 3′ end of the 5′-half vector and at the 5′ end of the 3′-half vector, dual AAV overlapping), which will mediate reconstitution of a single large genome by homologous recombination (Duan et al., 2001). This strategy depends on the recombinogenic properties of the transgene overlapping sequences (Ghosh et al., 2006). A third dual AAV strategy (hybrid) is based on adding a highly recombinogenic region from an exogenous gene (i.e., alkaline phosphatase; Ghosh et al., 2008, Ghosh et al., 2011)) to the trans-splicing vectors. The added region is placed downstream of the SD signal in the 5′-half vector and upstream of the SA signal in the 3′-half vector in order to increase recombination between the dual AAVs.

A “hybrid AAV” or “hybrid rAAV” refers to an rAAV genome packaged with a capsid of a different AAV serotype (and preferably, of a different serotype from the one or more AAV ITRs), and may otherwise be referred to as a pseudotyped rAAV. For example, an rAAV type 1, 2, 3, 4, 5, 6, 7, 8, 9, 10, 11, 12, 13, 14, 15, or 16 genome may be encapsidated within an AAV type 1, 2, 3, 4, 5, 6, 7, 8, 9, 10, 11, 12, 13, 14, 15, or 16 capsid or variants thereof, provided that the AAV capsid and genome (and preferably, the one or more AAV ITRs) are of different serotypes. In certain embodiments, a pseudotyped rAAV particle may be referred to as being of the type “x/y”, where “x” indicates the source of ITRs and “y” indicates the serotype of capsid, for example a 2/5 rAAV particle has ITRs from AAV2 and a capsid from AAV6.

In one illustrative embodiment, an AAV vector comprises one or more AAV ITRs and one or more capsid proteins from an AAV serotype selected from the group consisting of AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In one illustrative embodiment, an AAV vector comprises one or more AAV2 ITRs and one or more capsid proteins from an AAV serotype selected from the group consisting of AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

In one illustrative embodiment, an AAV vector comprises one or more AAV2 ITRs and one or more capsid proteins from an AAV serotype selected from the group consisting of AAV1, AAV5, AAV6, AAV6 (VP3 variant Y705F/Y731F/T492V), AAV8, AAV9, and AAV9 (VP3 variant Y731F).

In another illustrative embodiment, an AAV vector comprises one or more AAV2 ITRs and one or more capsid proteins from an AAV serotype selected from the group consisting of AAV6, AAV6 (VP3 variant Y705F/Y731F/T492V), AAV9, and AAV9 (VP3 variant Y731F).

In one illustrative embodiment, an AAV vector comprises one or more AAV2 ITRs and one or more capsid proteins from an AAV serotype selected from the group consisting of AAV9, and AAV9 (VP3 variant Y731F).

In one illustrative embodiment, an AAV vector comprises one or more AAV2 ITRs and one or more capsid proteins from an AAV serotype selected from the group consisting of AAV6 and AAV6 (VP3 variant Y705F/Y731F/T492V).

In one illustrative embodiment, an AAV vector comprises one or more AAV2 ITRs and one or more capsid proteins from an AAV6 serotype.

In one illustrative embodiment, an AAV vector comprises one or more AAV2 ITRs and one or more capsid proteins from an AAV6 (VP3 variant Y705F/Y731F/T492V) serotype.

A “host cell” includes cells transfected, infected, or transduced in vivo, ex vivo, or in vitro with a recombinant vector or a polynucleotide of the invention. Host cells may include virus producing cells and cells infected with viral vectors. In particular embodiments, host cells in vivo are infected with viral vector contemplated herein. In certain embodiments, the term “target cell” is used interchangeably with host cell and refers to infected cells of a desired cell type.

High titer AAV preparations can be produced using techniques known in the art, e.g., as described in U.S. Pat. Nos. 5,658,776; 6,566,118; 6,989,264; and 6,995,006; U.S. 2006/0188484; WO98/22607; WO2005/072364; and WO/1999/011764; and Viral Vectors for Gene Therapy: Methods and Protocols, ed. Machida, Humana Press, 2003; Samulski et al., (1989) J. Virology 63, 3822; Xiao et al., (1998) J. Virology 72, 2224; lnoue et al., (1998) J. Virol. 72, 7024. Methods of producing pseudotyped AAV vectors have also been reported (e.g., WO00/28004), as well as various modifications or formulations of AAV vectors, to reduce their immunogenicity upon in vivo administration (see e.g., WO01/23001; WO00/73316; WO04/112727; WO05/005610; WO99/06562).

2. Retroviral and Lentiviral Vectors

In particular embodiments, the vector is a retroviral vector or a lentiviral vector, in part since lentiviral vectors are capable of providing efficient delivery, integration and long term expression of transgenes into non-dividing cells both in vitro and in vivo. A variety of lentiviral vectors are known in the art, see Naldini et al., (1996a, 1996b, and 1998); Zufferey et al., (1997); Dull et al., 1998, U.S. Pat. Nos. 6,013,516; and 5,994,136, any of which may be adapted to produce a suitable vector for the compositions and methods contemplated herein.

In one embodiment, the lentiviral vector is an HIV vector.

In one embodiment, the lentiviral vector is a human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), caprine arthritis encephalitis virus (CAEV) and the like. HIV based vector backbones (i.e., HIV cis-acting sequence elements and HIV gag, pol and rev genes) are generally be preferred in connection with most aspects comprising lentiviral vectors as the HIV-based constructs are the most efficient at transduction of human cells.

The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (T) packaging signal, RRE), and/or other elements that increase therapeutic gene expression (e.g., poly (A) sequences), and a WPRE or HPRE.

One having ordinary skill in the art would recognize that the vector and compositions contemplated herein are not limited by any particular target sequence and that the genome editing platform could be designed to provide knockout/disruption or correction/insertion of any genomic locus where the sequence is known.

In various embodiments, any of the foregoing vector elements may be combined in various combinations and orientations. The skilled artisan would appreciate that many other different embodiments can be fashioned from the existing embodiments of the invention.

### F. Compositions and Formulations

The present invention further includes various pharmaceutical compositions comprising polynucleotides, vectors, and polypeptides contemplated herein and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible, including pharmaceutically acceptable cell culture media. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the vectors contemplated herein, use thereof in the pharmaceutical compositions of the invention is also contemplated.

The compositions of the invention may comprise one or more polypeptides, polynucleotides, and vectors comprising same, infected cells, etc., as described herein, formulated in pharmaceutically-acceptable or physiologically-acceptable solutions for administration to a cell or an animal, either alone, or in combination with one or more other modalities of therapy. It will also be understood that, if desired, the compositions of the invention may be administered in combination with other agents as well, such as, e.g., cytokines, e.g., anti-inflammatory cytokines, growth factors, hormones, small molecules or various pharmaceutically-active agents. There is virtually no limit to other components that may also be included in the compositions, provided that the additional agents do not adversely affect the ability of the composition to deliver the intended gene therapy.

In the pharmaceutical compositions contemplated herein, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intramuscular, intrathecal, intraneural, intraganglion, intracranial, and intraventricular administration and formulation.

In certain circumstances it will be desirable to deliver the compositions disclosed herein parenterally, intravenously, intramuscularly, intraperitoneally, intrathecally, intraneurally, intraganglionicly, intracranially, or intraventricularly. Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form should be sterile and should be fluid to the extent that easy syringability exists. It should be stable under the conditions of manufacture and storage and should be preserved against the contaminating action of microorganisms, such as bacteria and fungi. The carrier can be a solvent or dispersion medium containing, for example, water, ethanol, polyol (e.g., glycerol, propylene glycol, mannitol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabenes, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal intrathecal, intraneural, intraganglion, intracranial, and intraventricular administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington: The Science and Practice of Pharmacy, 20th Edition. Baltimore, Md.: Lippincott Williams & Wilkins, 2000). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions can be prepared by incorporating the active components in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, nanoparticles, exosomes, microparticles, microspheres, lipid particles, vesicles, optionally mixing with CPP polypeptides, and the like, for the introduction of the compositions of the present invention into suitable host cells. In particular, the compositions of the present invention may be formulated for delivery either encapsulated in a lipid particle, a liposome, an exosome, a vesicle, a nanosphere, a nanoparticle or the like. The formulation and use of such delivery vehicles can be carried out using known and conventional techniques. The formulations and compositions of the invention may comprise one or more polypeptides, polynucleotides, and small molecules, as described herein, formulated in pharmaceutically-acceptable or physiologically-acceptable solutions (e.g., culture medium) for administration to a cell or an animal, either alone, or in combination with one or more other modalities of therapy. It will also be understood that, if desired, the compositions of the invention may be administered in combination with other agents as well, such as, e.g., cells, other proteins or polypeptides or various pharmaceutically-active agents.

In a particular embodiment, a formulation or composition according to the present invention comprises a cell contacted with a combination of any number of polypeptides, polynucleotides, and viral vectors, as contemplated herein.

In certain aspects, the present invention provides formulations or compositions suitable for the delivery of viral vectors.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In certain aspects, the present invention provides pharmaceutically acceptable compositions which comprise a therapeutically-effective amount of one or more polynucleotides or polypeptides, as described herein, formulated together with one or more pharmaceutically acceptable carriers (additives) and/or diluents (e.g., pharmaceutically acceptable cell culture medium).

Particular embodiments of the invention may comprise other formulations, such as those that are well known in the pharmaceutical art, and are described, for example, in Remington: The Science and Practice of Pharmacy, 20th Edition. Baltimore, Md.: Lippincott Williams & Wilkins, 2000.

### G. Methods

As set forth herein, the present invention relates generally to polynucleotides, CRISPR-Cas systems, polynucleotides, vectors, genetically modified cells, and related compositions for use in gene therapy.

For example, in various embodiments, compositions, polynucleotides, or vectors contemplated herein comprise a complete genome editing platform and can be used to knockout or disrupt a gene or genetic regulatory sequence, correct a sequence in the genome, or insert genetic material into the genome in order to treat, prevent, ameliorate, or manage one or more symptoms or effects of a disease, disorder or condition in a subject in need. The compositions, polynucleotides, or vectors generally comprise one or more guide RNAs that function to target a CRISPR-Cas endonuclease to one or more target sites to facilitate altering the genome.

In various embodiments, for example, a composition, polynucleotide, or vector comprising a CRISPR-Cas endonuclease is administered to (or introduced into) one or more cell or tissue types of interest in order to disrupt or enable regulation of one or more genes of interest, such as a gene disclosed herein or a gene associated with a disease disclosed herein. For example, in certain more specific illustrative embodiments, a composition, polynucleotide, or vector comprising a CRISPR-Cas endonuclease is administered to (or introduced into) one or more nociceptive neuronal cells in order to disrupt or enable regulation of one or more nociceptive genes, including but not limited to voltage gated sodium channels, for the purpose of treating, preventing or ameliorating the effects of pain in a subject.

In certain embodiments, a method of genetically modifying a cell comprises introducing a composition, polynucleotide, or vector contemplated herein into the cell and inducing the expression of the switch polypeptide for a time sufficient to edit the genome of the cell. The type of cell genetically modified according to the methods of the invention can be essentially any type of cell associated with a disease or condition disclosed herein. In one embodiment, the cell is an excitatory cell. In one embodiment, the cell is a neuronal cell. In a particular embodiment, a nociceptive gene is disrupted to enable the treatment, prevent, or amelioration of pain. In one embodiment, the nociceptive gene is a voltage gated sodium channel. In a preferred embodiment the voltage gated sodium channel is selected from the group consisting of: Nav1.1, Nav1.3, Nav1.6, Nav1.7, Nav1.8, and Nav1.9.

In certain embodiments, the editing of the genome in a cell comprises insertion of a regulatable transcriptional regulatory element upstream of a transcription start site in a gene of the cell. The transcriptional regulatory element can be activated or repressed in the presence or absence of an exogenous ligand or small molecule.

In a particular embodiment, the regulatable transcriptional regulatory element is inserted upstream of a gene set forth in Table 2 or Table 3, thereby enabling the transcriptional control of the gene and facilitating the treatment, prevention, or amelioration of the symptoms or effects of the disease or condition associated with the gene.

In a particular embodiment, the regulatable transcriptional regulatory element is inserted upstream of a nociceptive gene thereby enabling the transcriptional control of the gene and facilitating the treatment, prevent, or amelioration of pain. In one embodiment, the nociceptive gene is a voltage gated ion channel, such as a voltage gated sodium or potassium channel. In a preferred embodiment the voltage gated ion channel is a voltage gated sodium channel selected from the group consisting of: Naᵥ1.1, Naᵥ1.3, Naᵥ1.6, Naᵥ1.7, Naᵥ1.8, and Naᵥ1.9.

In various embodiments, compositions, polynucleotides, or vectors contemplated herein comprising a complete CRISPR-Cas endonuclease genome editing platform are administered to (or introduced into) one or more neuronal cells that increase pain sensation or sensitivity to pain, e.g., nociceptor, peripheral sensory neurons, C-fibers, Aδ fibers, Aβ fibers, DRG neurons, TGG neurons, and the like. Editing or regulation of nociceptive genes decreases the pain sensation, decreases the sensitivity to pain and potentiates the analgesic effect of editing these neuronal cells. In a preferred embodiment the voltage gated ion channel employed is a voltage gated sodium channel selected from the group consisting of: Naᵥ1.1, Naᵥ1.3, Naᵥ1.6, Naᵥ1.7, Naᵥ1.8, and Naᵥ1.9.

Targeting the CRISPR-Cas endonuclease genome editing platform to a sub-population of nociceptors can be achieved, for example, by one or more of: selection of the vector (e.g., AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10); selection of a promoter; and delivery means. In particularly preferred embodiments, the compositions and methods contemplated herein are used in methods for effectively reducing pain in a subject in need thereof. Indeed, as will be apparent, much of the further description below is set out for purposes of illustration in relation to the treatment, prevention and/or management of pain in a subject. However, it will be understood that the same or similar strategies, methodologies and/or techniques can also be employed in the treatment of other diseases or conditions disclosed herein, including those set forth in Table 2 or Table 3.

In various embodiments, a method for controlling, managing, preventing, or treating pain in a subject comprises administering to the subject an effective amount of a composition, polynucleotide, or vector contemplated herein. In various embodiments, the vectors (e.g., viral vectors) are administered by direct injection to a cell, tissue, or organ of a subject in need of gene therapy, in vivo.

“Pain” refers to an uncomfortable feeling and/or an unpleasant sensation in the body of a subject. Feelings of pain can range from mild and occasional to severe and constant. Pain can be classified as acute pain or chronic pain.

Illustrative examples of pain that are amenable to treatment with the vectors, compositions, and methods contemplated herein, include but are not limited to acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, and menstrual pain.

Pain can be classified as acute or chronic. “Acute pain” refers to pain that begins suddenly and is usually sharp in quality. Acute pain might be mild and last just a moment, or it might be severe and last for weeks or months. In most cases, acute pain does not last longer than three months, and it disappears when the underlying cause of pain has been treated or has healed. Unrelieved acute pain, however, may lead to chronic pain. “Chronic pain” refers to ongoing or recurrent pain, lasting beyond the usual course of acute illness or injury or lasting for more than three to six months, and which adversely affects the individual's well-being. In particular embodiments, the term “chronic pain” refers to pain that continues when it should not. Chronic pain can be nociceptive pain or neuropathic pain.

In particular embodiments, the compositions and methods contemplated herein are effective in reducing acute pain.

In particular embodiments, the compositions and methods contemplated herein are effective in reducing chronic pain.

Clinical pain is present when discomfort and abnormal sensitivity feature among the patient's symptoms. Individuals can present with various pain symptoms. Such symptoms include: 1) spontaneous pain which may be dull, burning, or stabbing; 2) exaggerated pain responses to noxious stimuli (hyperalgesia); and 3) pain produced by normally innocuous stimuli (allodynia-Meyer et al., 1994, Textbook of Pain, 13-44). Although patients suffering from various forms of acute and chronic pain may have similar symptoms, the underlying mechanisms may be different and may, therefore, require different treatment strategies. Pain can also therefore be divided into a number of different subtypes according to differing pathophysiology, including nociceptive pain, inflammatory pain, and neuropathic pain.

In particular embodiments, the compositions and methods contemplated herein are effective in reducing nociceptive pain.

In particular embodiments, the compositions and methods contemplated herein are effective in reducing inflammatory pain.

In particular embodiments, the compositions and methods contemplated herein are effective in reducing neuropathic pain.

Nociceptive pain is induced by tissue injury or by intense stimuli with the potential to cause injury. Moderate to severe acute nociceptive pain is a prominent feature of pain from central nervous system trauma, strains/sprains, burns, myocardial infarction and acute pancreatitis, post-operative pain (pain following any type of surgical procedure), posttraumatic pain, renal colic, cancer pain and back pain. Cancer pain may be chronic pain such as tumor related pain (e.g., bone pain, headache, facial pain or visceral pain) or pain associated with cancer therapy (e.g., postchemotherapy syndrome, chronic postsurgical pain syndrome or post radiation syndrome). Cancer pain may also occur in response to chemotherapy, immunotherapy, hormonal therapy or radiotherapy. Back pain may be due to herniated or ruptured intervertebral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Neuropathic pain can be defined as pain initiated or caused by a primary lesion or dysfunction in the nervous system. Etiologies of neuropathic pain include, e.g., peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

Neuropathic pain can be related to a pain disorder, a term referring to a disease, disorder or condition associated with or caused by pain. Illustrative examples of pain disorders include arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

The inflammatory process is a complex series of biochemical and cellular events, activated in response to tissue injury or the presence of foreign substances, which results in swelling and pain. Arthritic pain is a common inflammatory pain.

Other types of pain that are amenable to treatment with the vectors, compositions, and methods contemplated herein, include but are not limited to pain resulting from musculoskeletal disorders, including myalgia, fibromyalgia, spondylitis, sero-negative (non-rheumatoid) arthropathies, non-articular rheumatism, dystrophinopathy, glycogenolysis, polymyositis and pyomyositis; heart and vascular pain, including pain caused by angina, myocardical infarction, mitral stenosis, pericarditis, Raynaud's phenomenon, scleredoma and skeletal muscle ischemia; head pain, such as migraine (including migraine with aura and migraine without aura), cluster headache, tension-type headache mixed headache and headache associated with vascular disorders; and orofacial pain, including dental pain, otic pain, burning mouth syndrome, and temporomandibular myofascial pain.

The ability of the compositions and methods contemplated herein to reduce the amount of pain experienced by a human subject can be determined using a variety of pain scales. Patient self-reporting can be used to assess whether pain is reduced; see, e.g., Katz and Melzack (1999) Surg. Clin. North Am. 79:231. Alternatively, an observational pain scale can be used. The LANSS Pain Scale can be used to assess whether pain is reduced; see, e.g., Bennett (2001) Pain 92:147. A visual analog pain scale can be used; see, e.g., Schmader (2002) Clin. J. Pain 18:350. The Likert pain scale can be used; e.g., where 0 is no pain, 5 is moderate pain, and 10 is the worst pain possible. Self-report pain scales for children include, e.g., Faces Pain Scale; Wong-Baker FACES Pain Rating Scale; and Colored Analog Scale. Self-report pain scales for adults include, e.g., Visual Analog Scale; Verbal Numerical Rating Scale; Verbal Descriptor Scale; and Brief Pain Inventory. Pain measurement scales include, e.g., Alder Hey Triage Pain Score (Stewart et al. (2004) Arch. Dis. Child. 89:625); Behavioral Pain Scale (Payen et al. (2001) Critical Care Medicine 29:2258); Brief Pain Inventory (Cleeland and Ryan (1994) Ann. Acad. Med. Singapore 23: 129); Checklist of Nonverbal Pain Indicators (Feldt (2000) Pain Manag. Nurs. 1: 13); Critical-Care Pain Observation Tool (Gelinas et al. (2006) Am. J. Crit. Care 15:420); COMFORT scale (Ambuel et al. (1992) J. Pediatric Psychol. 17:95); Dallas Pain Questionnaire (Ozguler et al. (2002) Spine 27:1783); Dolorimeter Pain Index (Hardy et al. (1952) Pain Sensations and Reactions Baltimore: The Williams & Wilkins Co.); Faces Pain Scale—Revised (Hicks et al. (2001) Pain 93:173); Face Legs Activity Cry Consolability Scale; McGill Pain Questionnaire (Melzack (1975) Pain 1:277); Descriptor Differential Scale (Gracely and Kwilosz (1988) Pain 35:279); Numerical 11 point Box (Jensen et al. (1989) Clin. J. Pain 5: 153); Numeric Rating Scale (Hartrick et al. (2003) Pain Pract. 3:310); Wong-Baker FACES Pain Rating Scale; and Visual Analog Scale (Huskisson (1982) J. Rheumatol. 9:768), In various embodiments, disruption or regulation of voltage gated sodium channels allows for the treatment, prevention, amelioration, or management associated with various channelopathies associated with the channels. In a preferred embodiment the voltage gated sodium channel is selected from the group consisting of: Naᵥ1.1, Naᵥ1.3, Naᵥ1.6, Naᵥ1.7, Naᵥ1.8, and Naᵥ1.9.

In further related embodiments, illustrative examples of channelopathies suitable for treatment with the compositions, polynucleotides and vectors contemplated herein include, but are not limited to: Channelopathy-associated Insensitivity to Pain (CIP), an extremely rare hereditary loss-of-function mutation of Naᵥ1.7; Primary Erythermalgia (PE), Fibromyalgia, and Paroxysmal Extreme Pain Disorder (PEPD), which result from from Naᵥ1.7 gain-of-function mutations; and Febrile Epilepsy, Generalized Epilepsy with Febrile Seizures, Dravet syndrome, West syndrome, Doose syndrome, Intractable Childhood Epilepsy with Generalized Tonic-Clonic seizures (ICEGTC), Panayiotopoulos syndrome, Familial Hemiplegic Migraine (FHM), Familial Autism, Rasmussen's Encephalitis, Lennox-Gastaut syndrome, Epilepsy, Pain, Hyperkalemic Periodic Paralysis, Paramyotonia Congenita, Potassium-Aggravated Myotonia, Long QT Syndrome, Brugada Syndrome, Idiopathic Ventricular Fibrillation, Irritable Bowel Syndrome, and Neuropsychiatric Disorders, which result from mutations in Naᵥ1.1, Naᵥ1.2, Naᵥ1.3, Naᵥ1.4, Naᵥ1.5, Naᵥ1.6, Naᵥ1.8, and Naᵥ1.9. Illustrative voltage-gated sodium channels and their associated disease indications are set forth in Table 2 below.

TABLE 2
Known voltage-gated sodium channels and associated indications

| Protein | Gene | Tissue Location | Indications |
|---|---|---|---|
| Naᵥ1.1 | SCN1A | CNS, PNS, heart | Epilepsy, GEFS+, Dravet syndrome (severe |
| | | | myoclonic epilepsy of infancy or SMEI), |
| | | | borderline SMEI (SMEB), West syndrome |
| | | | (infantile spasms), Doose syndrome |
| | | | (myoclonic astatic epilepsy), intractable |
| | | | childhood epilepsy with generalized tonic- |
| | | | clonic seizures (ICEGTC), Panayiotopouos |
| | | | syndrome, familial hemiplegic migraine |
| | | | (FMH), familial autism, Rasmussen's |
| | | | encephalitis, Lennox-Gastaut syndrome |
| Naᵥ1.2 | SCN2A | CNS, PNS | Inherited febrile seizures and epilepsy |
| Naᵥ1.3 | SCN3A | CNS, PNS, heart | Epilepsy, pain |
| Naᵥ1.4 | SCN4A | skeletal muscle | Hyperkalemic periodic paralysis, |
| | | | paramyotonia congenita, and potassium- |
| | | | aggravated myotonia |
| Naᵥ1.5 | SCN5A | heart, skeletal | Long QT syndrome, Brugada syndrome, |
| | | muscle, smooth | idiopathic ventricular fibrillation, irritable |
| | | muscle, CNS | bowel syndrome (IBS) |
| Naᵥ1.6 | SCN8A | CNS, PNS, heart | epilepsy |
| Naᵥ1.7 | SCN9A | PNS | Erythromelalgia, paroxysmal extreme pain |
| | | | disorder (PEPD), channelopathy-associated |
| | | | insensitivity to pain (CIP), fibromyalgia |
| Naᵥ1.8 | SCN10A | PNS | Pain, neuropsychiatric disorders |
| Naᵥ1.9 | SCN11A | PNS | Pain |
| Naₓ | SCN7A | cardiac, skeletal | None known |
| | | muscle, PNS | |

CNS: Central Nervous System,
PNS: Peripheral Nervous System including Dorsal Root Ganglion (DRG) and Trigeminal Ganglion (TGG) neurons

In one embodiment, the compositions, polynucleotides, and vectors contemplated herein are administered to a subject in order to disrupt or regulate the activity of Naᵥ1.7 to treat pain, e.g., chronic pain. Naᵥ1.7 gene function can be disrupted by multiple mechanisms including insertion of a treanscriptional regulatory element, excision of a fragment or the entire coding region, insertion of a mutation causing a premature stop codon, disruption of the promoter region, or introduction of mutations in specific loci that are associated with CIP. Naᵥ1.7 loci responsible for CIP that are suitable for editing with the compositions, polynucleotides, and vectors contemplated herein include, but are not limited to R277X, Y328X, S459X, E693X, I767X, R830X, R896Q, W897X, F1200LfsX33, I1235LfsX2, R1370_L1374del, c.4336-7_10delGTTT, R1488X, I1493SfsX8, W1689X, and K1659X.

In certain embodiments, the method provides a 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more reduction in the neuropathic pain in a subject compared to an untreated subject.

In particular embodiments, the vectors contemplated herein are administered or introduced into one or more neuronal cells. The neuronal cells may be the same type of neuronal cells, or a mixed population of different types of neuronal cells.

In one embodiment, the neuronal cell is a nociceptor or peripheral sensory neuron.

Illustrative examples of sensory neurons include, but are not limited to, dorsal root ganglion (DRG) neurons and trigeminal ganglion (TGG) neurons.

In one embodiment, the neuronal cell is an inhibitory interneuron involved in the neuronal pain circuit.

In particular embodiments, a vector is parenterally, intravenously, intramuscularly, intraperitoneally, intrathecally, intracranially, intraneurally, intraganglionicly, intraspinally, or intraventricularly administered to a subject in order to introduce the vector into one or more neuronal cells. In various embodiments, the vector is rAAV.

In one embodiment, AAV is administered to sensory neuron or nociceptor, e.g., DRG neurons, TGG neurons, etc. by intrathecal (IT) or intraganglionic (IG) administration.

The IT route delivers AAV to the cerebrospinal fluid (CSF). In animals, IT administration has been achieved by inserting an IT catheter through the cisterna magna and advancing it caudally to the lumbar level. In humans, IT delivery can be easily performed by lumbar puncture (LP), a routine bedside procedure with excellent safety profile.

The IG route delivers AAV directly into the DRG or TGG parenchyma. In animals IG administration to the DRG is performed by an open neurosurgical procedure that is not desirable in humans because it would require a complicated and invasive procedure. In humans, a minimally invasive, CT imaging-guided technique to safely target the DRG can be used. A customized needle assembly for convection enhanced delivery (CED) can be used to deliver AAV into the DRG parenchyma.

In still other more general embodiments of the invention, a method for treating, controlling, managing or preventing one or more symptoms or other effects in a subject of a disease or condition set forth in Table 3 below comprises administering to the subject an effective amount of a composition, polynucleotide, or vector contemplated herein. As will be understood, the selection of vectors, promoters, target sequences and/or other elements for use in the design, preparation and deployment of nucleic acids and vectors of the invention for the disease indications of Table 3 will be readily apparent to the skilled artisan in light of the present disclosure and the level of skill and knowledge in the art.

TABLE 3
Further Disease Indications and Associated Genes

| Disease | Protein | Gene |
|---|---|---|
| AADC Deficiency (global | Dopa decarboxylase | DDC |
| muscular hypotonia/dystonia) | | |
| Achromatopsia type 2 | Cyclic nucleotide-gated channel, α3 | CNGA3 |
| | subunit | |
| Achromatopsia type 3 | Cyclic nucleotide-gated channel, β3 | CNGB3 |
| | subunit | |
| Aland Island eye disease | Cav1.4: calcium channel, voltage- | CACNA1F |
| | gated, L type, α1F subunit | |
| Amyotrophic lateral sclerosis | Superoxide dismutase 1 | SOD1 |
| (monogenic) | | |
| Andersen-Tawil syndrome | Kir2.1: potassium channel, | KCNJ2 |
| | inwardly-rectifying, subfamily J, | |
| | member 2 | |
| Angelman syndrome | Ubiquitin protein ligase E3A | UBE3a |
| ATRX, autism | Alpha thalassemia/mental | ATRX |
| | retardation syndrome X-linked | |
| Batten Disease (neuronal ceroid | Tripeptidyl peptidase 1 | TPP1 |
| lipofuscinoses) | | |
| Batten Disease (neuronal ceroid | Palmitoly protein thioesterase 1 | PPT1 |
| lipofuscinoses) | | |
| Batten Disease (neuronal ceroid | Cathepsin D | CTSD |
| lipofuscinoses) | | |
| Batten Disease (neuronal ceroid | Cathepsin F | CTSF |
| lipofuscinoses) | | |
| Benign familial infantile epilepsy | Nav2.1: sodium channel, voltage- | SCN2A |
| | gated, type II, α subunit | |
| Benign familial neonatal epilepsy | Kv7.2: potassium channel, voltage- | KCNQ2 |
| | gated, KQT-like subfamily, member 2 | |
| Benign familial neonatal epilepsy | Kv7.3: potassium channel, voltage- | KCNQ3 |
| | gated, KQT-like subfamily, member 3 | |
| Bestrophinopathy, autosomal- | Bestrophin 1 | BEST1 |
| recessive | | |
| Central core disease | RyR1: ryanodine receptor 1 | RYR1 |
| Cerebral autosomal dominant | Neurogenic locus notch homolog | Notch 3 |
| arteriopathy with subcortical | protein 3 | |
| infarcts and leucoencephalopathy | | |
| (CADASIL) | | |
| Charcot-Marie-Tooth disease type | Transient receptor potential cation | TRPV4 |
| 2C | channel, subfamily V, member 4 | |
| Childhood absence epilepsy | γ-aminobutyric acid A receptor, α1 | GABRA1 |
| | subunit | |
| Childhood absence epilepsy | γ-aminobutyric acid A receptor, α6 | GABRA6 |
| | subunit | |
| Childhood absence epilepsy | γ-aminobutyric acid A receptor, β3 | GABRB3 |
| | subunit | |
| Childhood absence epilepsy | γ-aminobutyric acid A receptor, γ2 | GABRG2 |
| | subunit | |
| Childhood absence epilepsy | Cav3.2: calcium channel, voltage- | CACNA1H |
| | gated, T type, α1H subunit | |
| Coffin-Lowry X-linked mental- | Ribosomal Protein S6 Kinase A3 | RPS6KA3 |
| retardation syndrome | | |
| Cognitive impairment with or | Nav1.6: sodium channel, voltage- | SCN8A |
| without cerebellar ataxia | gated, type VIII, α subunit | |
| Cone-rod dystropy, X-linked, type 3 | Cav1.4: calcium channel, voltage- | CACNA1F |
| | gated, L type, α1F subunit | |
| Congenital distal spinal muscular | Transient receptor potential cation | TRPV4 |
| atrophy | channel, subfamily V, member 4 | |
| Congenital indifference to pain, | Nav1.7: Sodium channel, voltage- | SCN9A |
| autosomal-recessive | gated, type IX, α subunit | |
| Congenital myasthenic syndrome | Cholinergic receptor, muscle | CHRNA1 |
| | nicotinic, α1 subunit | |
| Congenital myasthenic syndrome | Cholinergic receptor, muscle | CHRNB1 |
| | nicotinic, β1 subunit | |
| Congenital myasthenic syndrome | Cholinergic receptor, muscle | CHRND |
| | nicotinic, δ subunit | |
| Congenital myasthenic syndrome | Cholinergic receptor, muscle | CHRNE |
| | nicotinic, ε subunit | |
| Congenital myasthenic syndrome | Nav1.4: sodium channel, voltage- | SCN4A |
| | gated, type IV, α subunit | |
| Congenital stationary night | Transient receptor potential cation | TRPM1 |
| blindness type 1C | channel, subfamily M, member 1 | |
| Congenital stationary night | Cav1.4: calcium channel, voltage- | CACNA1F |
| blindness type 2A | gated, L type, α1F subunit | |
| Deafness, autosomal-dominant, | Kv7.4: potassium channel, voltage- | KCNQ4 |
| type 2A | gated, KQT-like subfamily, member 4 | |
| Deafness, autosomal-recessive, | Kir4.1: potassium channel, | KCNJ10 |
| type 4, with enlarged | inwardly-rectifying, subfamily J, | |
| | member 10 | |
| Dravet syndrome | Nav1.1: sodium channel, voltage- | SCN1A |
| | gated, type I, α subunit | |
| Dravet syndrome | γ-aminobutyric acid A receptor, γ2 | GABRG2 |
| | subunit | |
| Early infantile epileptic | Nav2.1: sodium channel, voltage- | SCN2A |
| encephalopathy type 11 | gated, type II, α subunit | |
| Early infantile epileptic | Nav1.6: sodium channel, voltage- | SCN8A |
| encephalopathy type 13 | gated, type VIII, α subunit | |
| Early infantile epileptic | KCa4.1: potassium channel, | KCNT1 |
| encephalopathy type 14 | subfamily T, member 1 | |
| Early infantile epileptic | Kv7.2: potassium channel, voltage- | KCNQ2 |
| encephalopathy type 7 | gated, KQT-like subfamily, member 2 | |
| EAST/SeSAME syndrome | Kir4.1: potassium channel, | KCNJ10 |
| | inwardly-rectifying, subfamily J, | |
| | member 10 | |
| Episodic ataxia type 1 | Kv1.1: potassium channel, voltage- | KCNA1 |
| | gated, shaker-related subfamily, | |
| | member 1 | |
| Episodic ataxia type 2 | Cav2.1: calcium channel, voltage- | CACNA1A |
| | gated, P/Q type, α1A subunit | |
| Episodic ataxia type 5 | Cavβ4: calcium channel, voltage- | CACNB4 |
| | gated, β4 subunit | |
| Facioscapulohumeral (FSH) | D4Z4 repeat in the 4q35 | D4Z4, |
| muscular dystrophy | subtelomeric region of Chromosome | DUX4 |
| | 4, Double homeobox 4 | |
| Familial episodic pain syndrome | Transient receptor potential cation | TRPA1 |
| | channel, subfamily A, member 1 | |
| Familial hemiplegic migraine type 1 | Cav2.1: calcium channel, voltage- | CACNA1A |
| | gated, P/Q type, α1A subunit | |
| Familial hemiplegic migraine type 3 | Nav1.1: sodium channel, voltage- | SCN1A |
| | gated, type I, α subunit | |
| Friedreich's ataxia | Expansion of an intronic GAA | FXN |
| | triplet repeat in the Frataxin gene | |
| Generalized epilepsy with febrile | Navβ1: sodium channel, voltage- | SCN1B |
| seizures plus (GEFS+) | gated, type I, β subunit | |
| Generalized epilepsy with febrile | Nav1.1: sodium channel, voltage- | SCN1A |
| seizures plus (GEFS+) | gated, type I, α subunit | |
| Generalized epilepsy with febrile | γ-aminobutyric acid A receptor, γ2 | GABRG2 |
| seizures plus (GEFS+) | subunit | |
| Generalized epilepsy with | KCa1.1: potassium channel, | KCNMA1 |
| paroxysmal dyskinesia | calcium-activated, large | |
| | conductance, subfamily M, α1 | |
| | subunit | |
| Hereditary hyperekplexia | Glycine receptor, α1 subunit | GLRA1 |
| Hereditary hyperekplexia | Glycine receptor, β subunit | GLRB |
| Hereditary motor and sensory | CMT1 (Charcot-Marie-Tooth | PMP-22, |
| neuropathy (HMSN) | disease 1), CMT1A, CMT1B, | P0, LITAF, |
| | CMT1C, CMT1D, CMT1E | EGR2, |
| | | NEFL |
| Hereditary motor and sensory | CMT2 (Charcot-Marie-Tooth | Mitofusin |
| neuropathy (HMSN) | disease 2), CMT2A, CMT2B, | 2, 1B-b, |
| | CMT2D, CMT2E, CMT2H, CMT2I | RAB7, |
| | | GARS, |
| | | NEFL, |
| | | HSP27, |
| | | HSP22 |
| Hereditary motor and sensory | CMT3 (Charcot-Marie-Tooth | P0, PMP- |
| neuropathy (HMSN) | disease 3) | 22 |
| Hereditary motor and sensory | CMT4 (Charcot-Marie-Tooth | GDAP1 |
| neuropathy (HMSN) | disease 4) | (CMT4A), |
| | | MTMR13 |
| | | (CMT4B1), |
| | | MTMR2 |
| | | (CMT4B2), |
| | | SH3TC2 |
| | | (CMT4C), |
| | | NDG1 |
| | | (CMT4D), |
| | | EGR2 |
| | | (CMT4E), |
| | | PRX |
| | | (CMT4F), |
| | | FDG4 |
| | | (CMT4H), |
| | | and FIG4 |
| | | (CMT4J). |
| Hereditary motor and sensory | CMTX (Connexin-32 gene on the X | Cx-32 |
| neuropathy (HMSN) | chromosome) | |
| Hereditary neuropathy with | Peripheral myelin protein 22 | PMP22 |
| liability to pressure palsies | | |
| (HNLPP) | | |
| Hereditary sensory and autonomic | DNA methyltransferase 1 | DNMT1 |
| neuropathy type 1 with adult-onset | | |
| dementia; ADCA-DN | | |
| Hereditary spastic paraparesis | SPG1-SPG33 | numerous |
| (HSP) | | |
| Homocystinuria | Cystathionine beta synthase | CBS, |
| | deficiency | MTHFR, |
| | | MTR, |
| | | MTRR, |
| | | and |
| | | MMADHC |
| Huntington's disease (HD) | Huntingtin | HTT |
| Hyperkalemic periodic paralysis | Nav1.4: sodium channel, voltage- | SCN4A |
| | gated, type IV, α subunit | |
| Hypokalemic periodic paralysis | Cav1.1: calcium channel, voltage- | CACNA1S |
| type 1 | gated, L type, α1S subunit | |
| Hypokalemic periodic paralysis | Nav1.4: sodium channel, voltage- | SCN4A |
| type 2 | gated, type IV, α subunit | |
| ICF1 mental-retardation syndrome | DNA methyltransferase 3B | DNMT3B |
| ICF2 mental-retardation syndrome | Zinc Finger And BTB Domain | ZBTB24 |
| | Containing 24 | |
| Juvenile macular degeneration | Cyclic nucleotide-gated channel, β3 | CNGB3 |
| | subunit | |
| Juvenile myoclonic epilepsy | γ-aminobutyric acid A receptor, α1 | GABRA1 |
| | subunit | |
| Juvenile myoclonic epilepsy | Cavβ4: calcium channel, voltage- | CACNB4 |
| | gated, β4 subunit | |
| Kleefstra syndrome (mental | Histone methyltransferase 1 | EHMT1 |
| retardation); schizophrenia; | | |
| nonspecific psychiatric phenotypes | | |
| and neurodegenerative disease in | | |
| postadolescence period | | |
| Malignant hyperthermia | RyR1: ryanodine receptor 1 | RYR1 |
| susceptibility | | |
| Malignant hyperthermia | Cav1.1: calcium channel, voltage- | CACNA1S |
| susceptibility | gated, L type, α1S subunit | |
| Maple syrup urine disease | Branched Chain Keto Acid | BCKDHA, |
| (MSUD) | Dehydrogenase E1 | BCKDHB, |
| | | and DBT |
| McArdle's disease | Glycogen phosphorylase, muscle | PYGM |
| (myophosphorylase deficiency) | form | |
| Mucolipidosis type IV | TRPML1/mucolipin 1 | MCOLN1 |
| Multiple pterygium syndrome, | Cholinergic receptor, muscle | CHRNA1 |
| lethal type | nicotinic, α1 subunit | |
| Multiple pterygium syndrome, | Cholinergic receptor, muscle | CHRND |
| lethal type | nicotinic, δ subunit | |
| Multiple pterygium syndrome, | Cholinergic receptor, muscle | CHRNG |
| lethal type | nicotinic, γ subunit | |
| Multiple pterygium syndrome, | Cholinergic receptor, muscle | CHRNG |
| nonlethal type (Escobar variant) | nicotinic, γ subunit | |
| Myotonia congenita, autosomal- | ClC-1: chloride channel 1, voltage- | CLCN1 |
| dominant (Thomsen disease) | gated | |
| Myotonia congenita, autosomal- | ClC-1: chloride channel 1, voltage- | CLCN1 |
| recessive (Becker disease) | gated | |
| Myotonic dystrophy type 1 | Myotonin-protein kinase | DMPK |
| Myotonic dystrophy type 2 | CCHC-type zinc finger nucleic acid | CNBP |
| | binding protein | |
| Neurofibromatosis | Neurofibromin 1, >1000 different | NF1 |
| | mutations in NF1 | |
| Nocturnal frontal lobe epilepsy | Cholinergic receptor, neuronal | CHRNA4 |
| type 1 | nicotinic, α4 subunit | |
| Nocturnal frontal lobe epilepsy | Cholinergic receptor, neuronal | CHRNB2 |
| type 3 | nicotinic, β2 subunit | |
| Nocturnal frontal lobe epilepsy | Cholinergic receptor, neuronal | CHRNA2 |
| type 4 | nicotinic, α2 subunit | |
| Nocturnal frontal lobe epilepsy | KCa4.1: potassium channel, | KCNT1 |
| type 5 | subfamily T, member 1 | |
| Oculopharyngeal muscular | Branched Chain Keto Acid | BCKDHA, |
| dystrophy (OPMD) | Dehydrogenase E1 | BCKDHB, |
| | | DBT, DLD |
| Paramyotonia congenita | Nav1.4: sodium channel, voltage- | SCN4A |
| | gated, type IV, α subunit | |
| Paroxysmal extreme pain disorder | Nav1.7: Sodium channel, voltage- | SCN9A |
| | gated, type IX, α subunit | |
| Potassium-aggravated myotonia | Nav1.4: sodium channel, voltage- | SCN4A |
| | gated, type IV, α subunit | |
| Primary erythermalgia | Nav1.7: sodium channel, voltage- | SCN9A |
| | gated, type IX, α subunit | |
| Retinitis pigmentosa type 45, | Cyclic nucleotide-gated channel, β1 | CNGB1 |
| autosomal-recessive | subunit | |
| Retinitis pigmentosa type 49, | Cyclic nucleotide-gated channel, α1 | CNGA1 |
| autosomal-recessive | subunit | |
| Retinitis pigmentosa type 50, | Bestrophin 1 | BEST1 |
| autosomal-dominant | | |
| RTT and other | Methyl-CpG-binding protein | MECP2 |
| neurodevelopmental syndromes; | | |
| autism | | |
| Rubinstein-Taybi syndrome | Branched-chain alpha-keto acid | CREBBP |
| (RSTS) 1 and 2 | dehydrogenase complex | |
| Scapuloperoneal spinal muscular | Transient receptor potential cation | TRPV4 |
| atrophy | channel, subfamily V, member 4 | |
| Small fiber neuropathy | Nav1.7: sodium channel, voltage- | SCN9A |
| | gated, type IX, α subunit | |
| Sotos syndrome (mental | Nuclear receptor SET-domain- | NSD1 |
| retardation) | containing protein | |
| Spinal muscular atrophy (SMA) | Survival motor neuron 1 | SMN1 |
| Spinal and bulbar muscular | Androgen receptor | AR |
| atrophy (SBMA) | | |
| Spinocerebellar ataxia type 13 | Kv3.3: potassium channel, voltage- | KCNC3 |
| | gated, Shaw-related subfamily, | |
| | member 3 | |
| Spinocerebellar ataxia type 6 | Cav2.1: calcium channel, voltage- | CACNA1A |
| | gated, P/Q type, α1A subunit | |
| Unverricht-Lundborg disease | Cystatin B | CSTB |
| Vitelliform macular dystrophy | Bestrophin 1 | BEST1 |
| Vitreoretinochoroidopathy | Bestrophin 1 | BEST1 |
| X-linked adrenoleukodystrophy | Adrenoleukodystrophy protein | ABCD1 |
| (X-ALD), adrenomyeloneuropathy | (ALDP) | |
| (AMN) | | |
| X-linked mental retardation | PHD Finger Protein 8 | PHF8 |
| without cleft lip and/or palate | | |
| (Siderius-Hamel) | | |
| X-linked mental retardation; | Lysine Demethylase 5C | KDM5C |
| autism | | |

In particular embodiments, a vector contemplated herein is administered to a subject at a titer of at least about 1×10⁹ genome particles/mL, at least about 1×10¹⁰ genome particles/mL, at least about 5×10¹⁰ genome particles/mL, at least about 1×10¹¹ genome particles/mL, at least about 5×10¹¹ genome particles/mL, at least about 1×10¹² genome particles/mL, at least about 5×10¹² genome particles/mL, at least about 6×10¹² genome particles/mL, at least about 7×10¹² genome particles/mL, at least about 8×10¹² genome particles/mL, at least about 9×10¹² genome particles/mL, at least about 10×10¹² genome particles/mL, at least about 15×10¹² genome particles/mL, at least about 20×10¹² genome particles/mL, at least about 25×10¹² genome particles/mL, at least about 50×10¹² genome particles/mL, or at least about 100×10¹² genome particles/mL. The terms “genome particles (gp),” or “genome equivalents,” or “genome copies” (gc) as used in reference to a viral titer, refer to the number of virions containing the recombinant AAV DNA genome, regardless of infectivity or functionality. The number of genome particles in a particular vector preparation can be measured by procedures such as described in the Examples herein, or for example, in Clark et al. (1999) Hum. Gene Ther., 10:1031-1039; Veldwijk et al. (2002) Mol. Ther., 6:272-278

In particular embodiments, a vector contemplated herein is administered to a subject at a titer of at least about 5×10⁹ infectious units/mL, at least about 6×10⁹ infectious units/mL, at least about 7×10⁹ infectious units/mL, at least about 8×10⁹ infectious units/mL, at least about 9×10⁹ infectious units/mL, at least about 10×10⁹ infectious units/mL, at least about 15×10⁹ infectious units/mL, at least about 20×10⁹ infectious units/mL, at least about 25×10⁹ infectious units/mL, at least about 50×10⁹ infectious units/mL, or at least about 100×10⁹ infectious units/mL. The terms “infection unit (iu),” “infectious particle,” or “replication unit,” as used in reference to a viral titer, refer to the number of infectious and replication-competent recombinant AAV vector particles as measured by the infectious center assay, also known as replication center assay, as described, for example, in McLaughlin et al. (1988) J. Virol., 62:1963-1973.

In particular embodiments, a vector contemplated herein is administered to a subject at a titer of at least about 5×10¹⁰ transducing units/mL, at least about 6×10¹⁰ transducing units/mL, at least about 7×10¹⁰ transducing units/mL, at least about 8×10¹⁰ transducing units/mL, at least about 9×10¹⁰ transducing units/mL, at least about 10×10¹⁰ transducing units/mL, at least about 15×10¹⁰ transducing units/mL, at least about 20×10¹⁰ transducing units/mL, at least about 25×10¹⁰ transducing units/mL, at least about 50×10¹⁰ transducing units/mL, or at least about 100×10¹⁰ transducing units/mL. The term “transducing unit (tu)” as used in reference to a viral titer, refers to the number of infectious recombinant AAV vector particles that result in the production of a functional transgene product as measured in functional assays such as described in Examples herein, or for example, in Xiao et al. (1997) Exp. Neurobiol., 144:113-124; or in Fisher et al. (1996) J. Virol., 70:520-532 (LFU assay).

### H. Kits

Compositions and reagents useful for the present invention may be packaged in kits to facilitate application of particular embodiments of the present invention. In some embodiments, a kit is provided comprising a polynucleotide, vector, or composition contemplated herein. In one embodiment, the kit comprises a recombinant virus contemplated herein. Embodiments of the kit contemplated herein may also comprised instructions. The instructions could be in any desired form, including but not limited to, printed on a kit insert, printed on one or more containers, as well as electronically stored instructions provided on an electronic storage medium, such as a computer readable storage medium.

The present invention now will be described more fully by the following examples. This invention may, however, be embodied in many different forms and should not be construed as limited to the embodiments set forth herein; rather, these embodiments are provided so that this disclosure will be thorough and complete, and will fully convey the scope of the invention to those skilled in the art.

### EXAMPLES

### Example 1: Treatment of a Patient Suffering from Chronic Pain

A patient suffering from chronic pain is treated using the compositions and methods disclosed herein. The patient is treated with 10¹⁵ vector genomes of AAV-hSYN1-Cas9 in a volume of 12.0 mL delivered into the subarachnoid space of the spinal cord (i.e., intrathecal). In this example, the AAV vector encodes the CRISPR Cas9 endonuclease derived from Streptococcus pyogenes under the control of the human Synapsin-1 (SYN1) promoter for selective neuronal expression (e.g. FIG. 1). The vector also contains an H1 promoter expressing a crRNA-trRNA fusion, with crRNA targeted to the Naᵥ1.7 (SNC9A gene) voltage gated sodium channel. The patient experiences chronic pain relief within approximately 1 week of vector administration resulting from disruption of Naᵥ1.7 channel function.

### Example 2: Treatment of a Patient Suffering from Chronic Pain

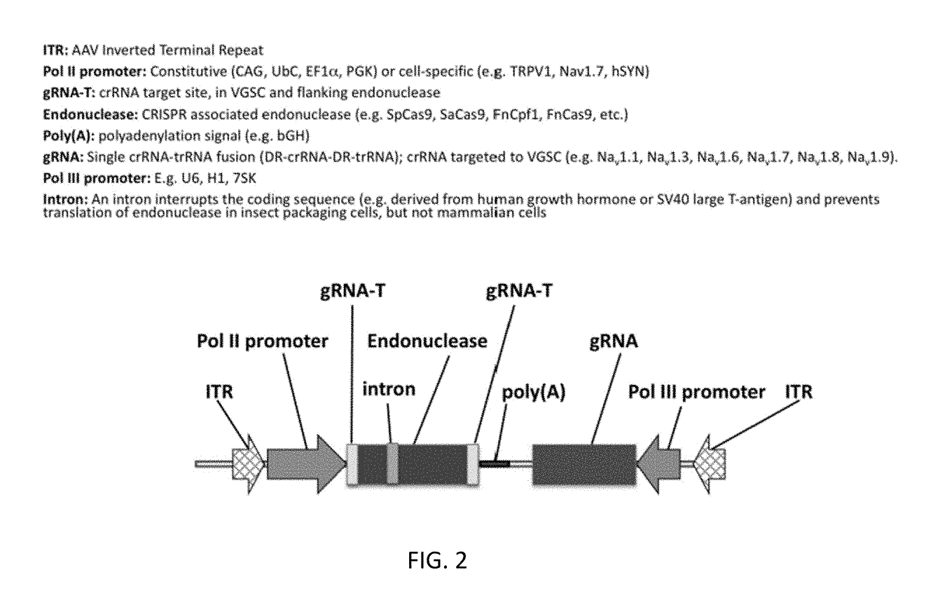

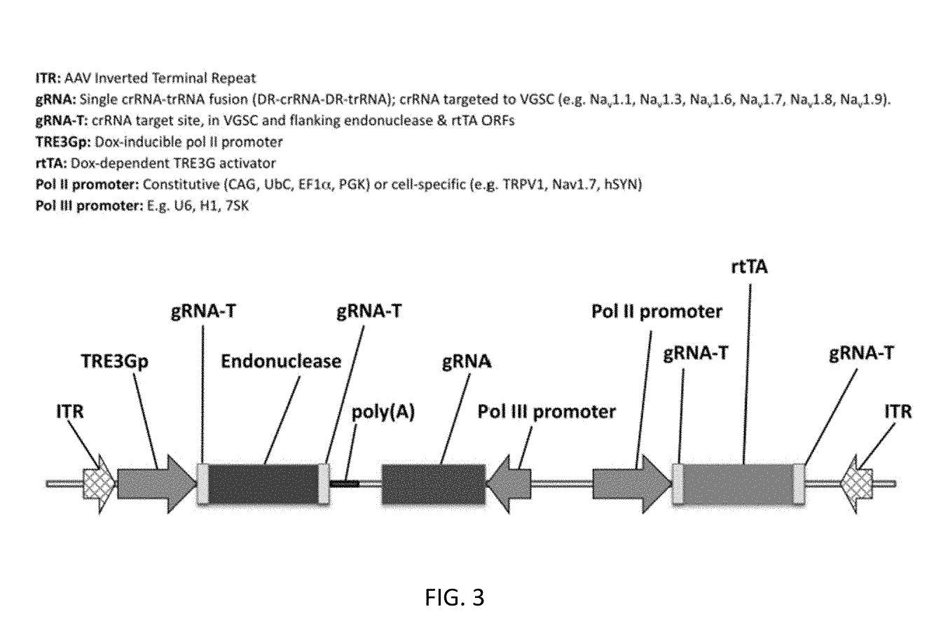

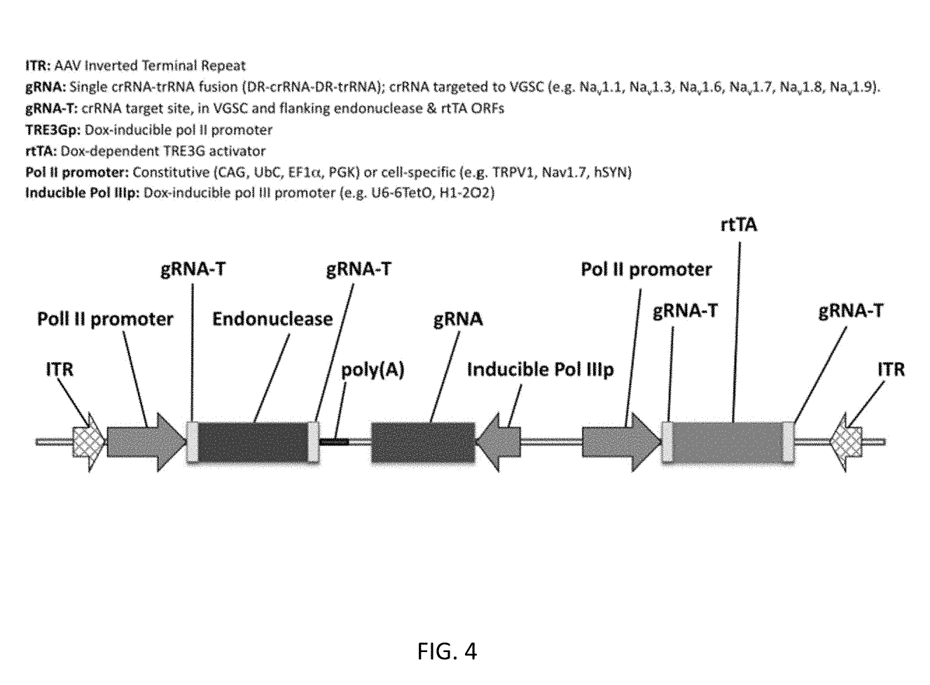

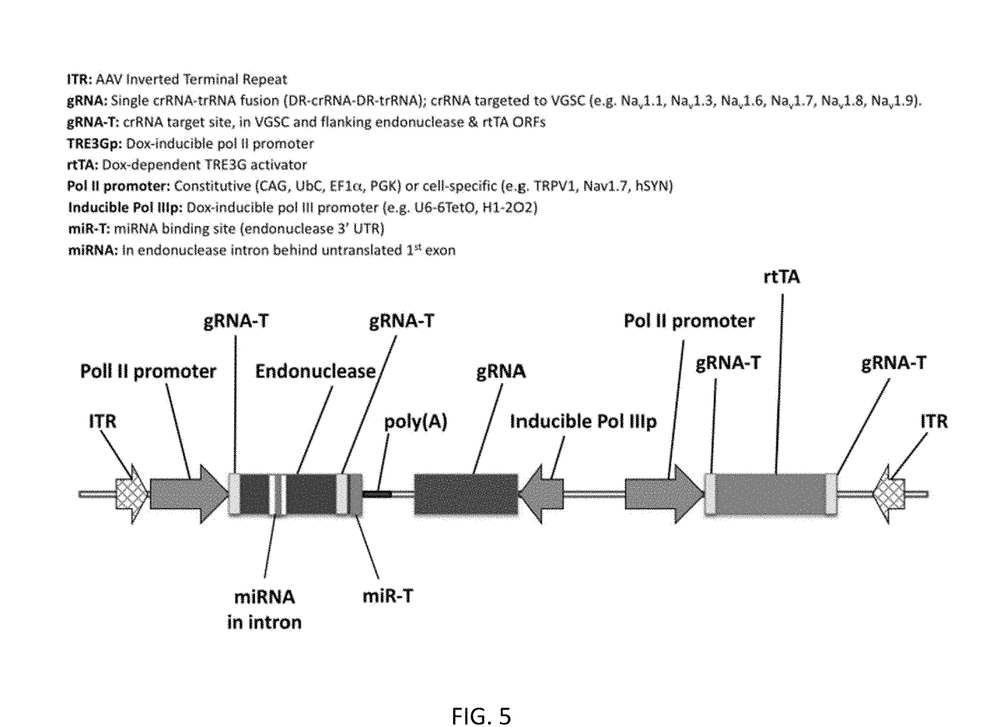

In a non-limiting example, a patient suffering from chronic radicular pain is treated using the compositions and methods disclosed herein. The patient is treated with 10¹³ vector genomes of AAV-hSYN1-Cpf1 in a volume of 1.0 mL delivered directly into one or more dorsal root ganglia (i.e., intraganglionic convection-enhanced delivery into lumbar, cervical, or thoracic DRGs). The specific DRGs responsible for signalling chronic pain are identified through a diagnostic selective nerve root block (e.g. lidocaine injection). In this example, the AAV vector encodes a transiently expressed CRISPR Cpf1 endonuclease derived from Francisella novicida flanked by gRNA target sites under transcriptional control of the human Synapsin-1 (SYN1) promoter for selective neuronal expression (e.g. FIG. 2-5). The vector also contains an H1 promoter expressing a crRNA-trRNA fusion, with crRNA targeted to the Naᵥ1.7 (SNC9A gene) voltage gated sodium channel. Following transduction of targeted DRG neurons, expression of Cpf1 occurs only transiently until disruption by the CRISPR-gRNA complex expressed by this vector. Upon distruption of Naᵥ1.7 channel function by the same CRISPR-gRNA complex, the patient experiences chronic pain relief within approximately 1 week of vector administration.

### Example 3: Treatment of a Patient Suffering from Chronic Pain

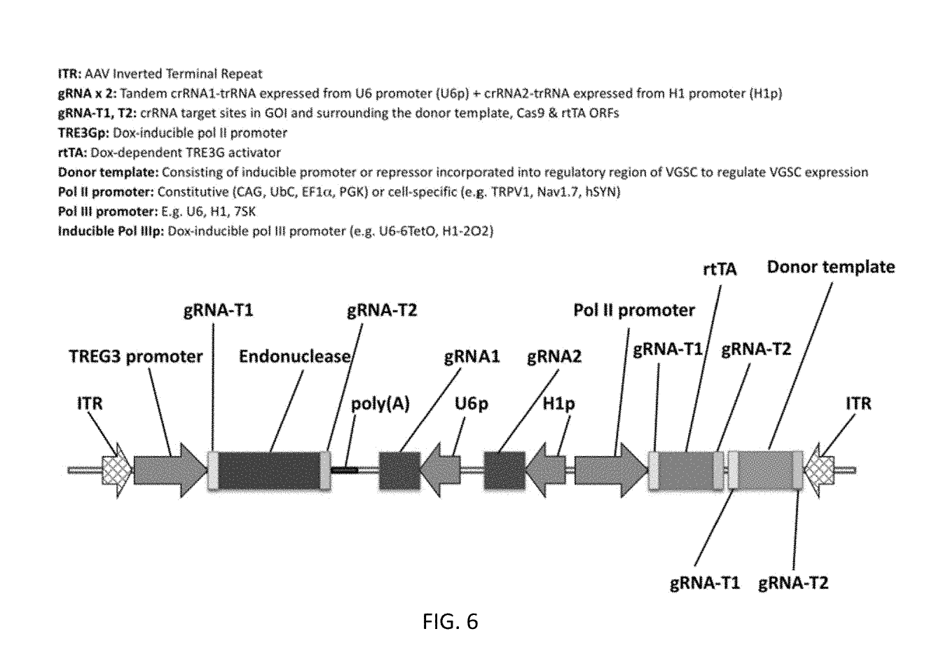

In another non-limiting example, a patient suffering from Trigeminal Neuralgia is treated using the compositions and methods disclosed herein. The patient is treated with 10¹³ vector genomes of AAV-hSYN1-Cpf1 in a volume of 1.0 mL delivered directly into one or both Trigeminal Ganglia (TGG). In this example, the AAV vector encodes a transiently expressed CRISPR Cpf1 endonuclease derived from Francisella novicida flanked by gRNA target sites under transcriptional control of the dox-inducible TRE3Gp promoter for transient expression (e.g. FIG. 6). The vector also contains H1 and U6 promoters expressing two unique gRNAs, with crRNAs targeted to disrupt Cpf1, rtTA, and the upstream regulatory region of the Naᵥ1.7 (SNC9A gene) voltage gated sodium channel. Lastly, the vector cotains a donor template sequence consisting of the inducible PPAR-γ promoter which is exogenously regulated by administration of the FDA approved small molecule rosiglitazone (Avandia). Following transduction of targeted TGG neurons, expression of Cpf1/rtTA occurs only transiently until disruption by the CRISPR-gRNA complex expressed by this vector. Following insertion of the PPAR-γ promoter donor template, gene expression levels of Naᵥ1.7 are modulated (up or down) upon oral administration of rosiglitazone, resulting in the patient experiencing chronic pain relief within approximately 1 week of vector administration and rosiglitazone administration.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A nucleic acid comprising a CRISPR-Cas system for the treatment, prevention, or amelioration of a disease or condition selected from the group consisting of chronic pain, a disease or condition set forth in Table 2, and a disease or condition set forth in Table 3.

2. A nucleic acid comprising an inducibly and transiently regulatable CRISPR-Cas system for the treatment, prevention, or amelioration of a disease or condition selected from the group consisting of chronic pain, a disease or condition set forth in Table 2, and a disease or condition set forth in Table 3.

3. The nucleic acid of claim 1 or claim 2, comprising
a) a first expression cassette that comprises an RNA polymerase II promoter operably linked to a polynucleotide encoding a CRISPR-Cas endonuclease; and
b) a second expression cassette that comprises one or more RNA polymerase III promoters each operably linked to a polynucleotide encoding one or more guide RNAs.

4. The nucleic acid of claim 1 or claim 2, comprising
a) a first expression cassette that comprises at least one regulatory element for transient expression and an RNA polymerase II promoter operably linked to a polynucleotide encoding a CRISPR-Cas endonuclease; and
b) a second expression cassette that comprises one or more RNA polymerase III promoters each operably linked to a polynucleotide encoding one or more guide RNAs.

5. The nucleic acid of claim 1 or claim 2, comprising
a) a first expression cassette that comprises at least one regulatory element for inducible expression and at least one regulatory element for transient expression and a polynucleotide encoding a CRISPR-Cas endonuclease;
b) a second expression cassette that comprises one or more RNA polymerase III promoters each operably linked to a polynucleotide encoding one or more guide RNAs; and
c) a third expression cassette that comprises at least one regulatory element for transient expression and an RNA polymerase II promoter operably linked to a polynucleotide encoding a switch polypeptide, wherein the switch polypeptide binds to the at least one element for inducible expression.

6. The nucleic acid of claim 1 or claim 2, comprising
a) a first expression cassette that comprises at least one regulatory element for transient expression and an RNA polymerase II promoter operably linked to a polynucleotide encoding a CRISPR-Cas endonuclease;
b) a second expression cassette that comprises at least one regulatory element for inducible expression operably linked to a polynucleotide encoding one or more guide RNAs; and
c) a third expression cassette that comprises at least one regulatory element for transient expression and an RNA polymerase II promoter operably linked to a polynucleotide encoding a switch polypeptide, wherein the switch polypeptide binds to the at least one element for inducible expression.

7. The nucleic acid of claim 1 or claim 2, comprising
a) a first expression cassette that comprises at least one regulatory element for inducible expression and at least one regulatory element for transient expression and a polynucleotide encoding a CRISPR-Cas endonuclease;
b) a second expression cassette that comprises one or more RNA polymerase III promoters each operably linked to one or more guide RNAs; and
c) a third expression cassette that comprises at least one regulatory element for transient expression and an RNA polymerase II promoter operably linked to a polynucleotide encoding a switch polypeptide, wherein the switch polypeptide binds to the at least one element for inducible expression.

8. The nucleic acid of any one of the preceding claims, wherein the at least one regulatory element for transient expression comprises one or more guide RNA target sites.

9. The nucleic acid of any one of the preceding claims, wherein the at least one regulatory element for transient expression comprises one or more guide RNA target sites and wherein the polynucleotide encoding the CRISPR-Cas endonuclease is flanked by the one or more guide RNA target sites.

10. The nucleic acid of any one of the preceding claims, wherein the at least one regulatory element for transient expression comprises one or more guide RNA target sites and wherein the polynucleotide encoding the switch polypeptide is flanked by one or more guide RNA target sites.

11. The nucleic acid of any one of the preceding claims, further comprising a polynucleotide encoding a template for altering at least one site in a genome that is flanked by one or more guide RNA target sites.

12. The nucleic acid of any one of claims 8-11, wherein the guide RNA target sites flanking any one of the polynucleotide encoding the CRISPR-Cas endonuclease, the polynucleotide encoding the switch polypeptide, and the polynucleotide encoding the template for altering at least one site in the genome are the same.

13. The nucleic acid of any one of claims 8-11, wherein the guide RNA target sites flanking the polynucleotide encoding the CRISPR-Cas endonuclease, the polynucleotide encoding the switch polypeptide, and the polynucleotide encoding the template for altering at least one site in the genome are the same.

14. The nucleic acid of any one of claims 8-11, wherein each of the guide RNA target sites flanking the 5′ end of the polynucleotide encoding the CRISPR-Cas endonuclease, the polynucleotide encoding the switch polypeptide, and the polynucleotide encoding the template for altering at least one site in the genome are the same.

15. The nucleic acid of any one of claims 8-11, wherein each of the guide RNA target site flanking the 3′ end of the polynucleotide encoding the CRISPR-Cas endonuclease, the polynucleotide encoding the switch polypeptide, and the polynucleotide encoding the template for altering at least one site in the genome are the same.

16. The nucleic acid of claim 14 or 15, wherein the guide RNA target site flanking the 5′ end and the guide RNA target site flanking the 3′ end of any one of the polynucleotides encoding the CRISPR-Cas endonuclease, the switch polypeptide, and the template for altering at least one site in the genome are different.

17. The nucleic acid of claim 14 or 15, wherein the guide RNA target site flanking the 5′ end and the guide RNA target site flanking the 3′ end of the polynucleotides encoding the CRISPR-Cas endonuclease, the switch polypeptide, and the template for altering at least one site in the genome are different.

18. The nucleic acid of any one of claims 14-17, wherein each of the guide RNAs target sites flanking the 5′ end of the polynucleotides encoding the CRISPR-Cas endonuclease, the switch polypeptide, and the template for altering at least one site in the genome are the same; wherein each of the guide RNAs target sites flanking the 3′ end of the polynucleotides encoding the CRISPR-Cas endonuclease, the switch polypeptide, and the template for altering at least one site in the genome are the same; and wherein the guide RNA target site flanking the 5′ end each polynucleotide is different from the guide RNA target site flanking the 3′ end of each of polynucleotide.

19. The nucleic acid of any one of claims 8-18, wherein the one or more guide RNA target sites in the vector are identical to one or more guide RNA target sites in the genome.

20. The nucleic acid of any one of claims 8-18, wherein the guide RNA target site flanking the 5′ end of each polynucleotide is identical to a guide RNA target site in the genome; wherein the guide RNA target site flanking the 3′ end of each polynucleotide is identical to a guide RNA target site in the genome; and wherein the guide RNA target site flanking the 5′ end each polynucleotide is different from the guide RNA target site flanking the 3′ end of each of polynucleotide.

21. The nucleic acid of any one of claims 1-20, wherein the one or more guide RNAs recognize and bind to each of the one or more guide RNAs target sites of any one of claims 8-20.

22. The nucleic acid of claim 21, wherein the vector comprises a single guide RNA that recognizes and binds all of the one or more guide RNA target sites of any one of claims 8-20.

23. The nucleic acid of claim 21, wherein the second expression cassette comprises a plurality of guide RNAs, wherein each of the plurality of guide RNAs recognizes and binds to one of the one or more guide RNA target sites of any one of claims 8-20.

24. The nucleic acid of any one of the preceding claims, wherein at least one RNA polymerase II promoter is a ubiquitous promoter, optionally wherein each RNA polymerase II promoter is a ubiquitous promoter, optionally wherein each ubiquitous promoter is different.

25. The nucleic acid of any one of the preceding claims, wherein the switch polypeptide is selected from the group consisting of a reverse tetracycline-controlled transactivator protein (rtTA), an ecdysone receptor, an estrogen receptor, a glucocorticoid receptor, a Hydrogen peroxide-inducible genes activator (oxyR) polypeptide, CymR polypeptide, and variants thereof.

26. The nucleic acid of claim 24 or claim 25, wherein the ubiquitous promoter is independently selected from the group consisting of: a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477-1482 (2007)), a Ubiquitin C promoter (UBC), a phosphoglycerate kinase-1 (PGK) promoter, and a cytomegalovirus enhancer/chicken β-actin (CAG) promoter.

27. The nucleic acid of claim 24 or claim 25, wherein the RNA polymerase II promoter is a tissue-specific or lineage-specific promoter.

28. The nucleic acid of claim 24 or claim 25, wherein the tissue-specific or lineage-specific promoter is selected from the group consisting of: a neuron specific promoter, a promoter operable in a trigeminal ganglion (TGG) neuron, a dorsal root ganglion (DRG) neuron, an hSYN1 promoter, a calcium/calmodulin-dependent protein kinase II a promoter, a tubulin alpha I promoter, a neuron-specific enolase promoter, a platelet-derived growth factor beta chain promoter, TRPV1 promoter, a Naᵥ1.7 promoter, a Naᵥ1.8 promoter, a Naᵥ1.9 promoter, or an Advillin promoter.

29. The nucleic acid of any one of the preceding claims, wherein at least one regulatory element for inducible expression is selected from the group consisting of: a tetracycline responsive promoter, an ecdysone responsive promoter, a cumate responsive promoter, a glucocorticoid responsive promoter, an estrogen responsive promoter, an RU-486 responsive promoter, a PPAR-γ promoter, and a peroxide inducible promoter.

30. The nucleic acid of any one of the preceding claims, wherein the one or more RNA polymerase III promoters is selected from the group consisting of: a human U6 snRNA promoter, a mouse U6 snRNA promoter, a human H1 RNA promoter, a mouse H1 RNA promoter, and a human tRNA-val promoter.

31. The nucleic acid of any one of the preceding claims, wherein the one or more RNA polymerase III promoters is independently selected from the group consisting of: a human U6 snRNA promoter, a mouse U6 snRNA promoter, a human H1 RNA promoter, a mouse H1 RNA promoter, and a human tRNA-val promoter.

32. The nucleic acid of any one of the preceding claims, wherein the CRISPR-Cas endonuclease selected from the group consisting of: Cpf1, Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

33. The nucleic acid of any one of the preceding claims, wherein the CRISPR-Cas endonuclease comprises a Cas9 polypeptide.

34. The nucleic acid of claim 33, wherein the Cas9 polypeptide is isolated from Staphylococcus aureus, Streptococcus pyogenes, Streptococcus thermophilis, Treponema denticola, and Neisseria meningitidis.

35. The nucleic acid of any one of claims 32-34, wherein the Cas9 polypeptide comprises one or more mutations in a HNH or a RuvC-like endonuclease domain or the HNH and the RuvC-like endonuclease domains.

36. The nucleic acid of claim 35, wherein the mutant Cas9 polypeptide is a nickase.

37. The nucleic acid of claim 35, wherein the mutant Cas9 polypeptide sequence is from Streptococcus pyogenes and comprises a mutation in the RuvC domain.

38. The nucleic acid of claim 37, wherein the mutation is a D10A mutation.

39. The nucleic acid of claim 35, wherein the mutant Cas9 polypeptide sequence is from Streptococcus pyogenes and comprises a mutation in the HNH domain.

40. The nucleic acid of claim 39, wherein the mutation is a D839A, H840A, or N863A mutation.

41. The nucleic acid of claim 35, wherein the mutant Cas9 polypeptide sequence is from Streptococcus thermophilis and comprises a mutation in the RuvC-like domain.

42. The nucleic acid of claim 41, wherein the mutation is a D9A mutation.

43. The nucleic acid of claim 35, wherein the mutant Cas9 polypeptide sequence is from Streptococcus thermophilis and comprises a mutation in the HNH domain.

44. The nucleic acid of claim 43, wherein the mutation is a D598A, H599A, or N622A mutation.

45. The nucleic acid of claim 35, wherein the mutant Cas9 polypeptide sequence is from Treponema denticola and comprises a mutation in the RuvC-like domain.

46. The nucleic acid of claim 45, wherein the mutation is a D13A mutation.

47. The nucleic acid of claim 35, wherein the mutant Cas9 polypeptide sequence is from Treponema denticola and comprises a mutation in the HNH domain.

48. The nucleic acid of claim 47, wherein the mutation is a D878A, H879A, or N902A mutation.

49. The nucleic acid of claim 35, wherein the mutant Cas9 polypeptide sequence is from Neisseria meningitidis and comprises a mutation in the RuvC domain.

50. The nucleic acid of claim 49, wherein the mutation is a D16A mutation.

51. The nucleic acid of claim 35, wherein the mutant Cas9 polypeptide sequence is from Neisseria meningitidis and comprises a mutation in the HNH domain.

52. The nucleic acid of claim 51, wherein the mutation is a D587A, H588A, or N611A mutation.

53. The nucleic acid of claim 35, wherein the mutant Cas9 polypeptide sequence is from Staphylococcus aureus and comprises a mutation in the RuvC domain.

54. The nucleic acid of claim 53, wherein the mutation is a D10A mutation.

55. The nucleic acid of claim 35, wherein the mutant Cas9 polypeptide sequence is from Staphylococcus aureus and comprises a mutation in the HNH domain.

56. The nucleic acid of claim 55, wherein the mutation is a N580A mutation.

57. The nucleic acid of any one of claims 32 to 56, wherein the Cas9 is a human codon optimized Cas9.

58. The nucleic acid of any one of claims 1 to 31, wherein the CRISPR-Cas endonuclease is a Cpf1 polypeptide.

59. The nucleic acid of claim 58, wherein the first expression cassette comprises a polynucleotide encoding a Cpf1 polypeptide isolated from Francisella novicida, Acidaminococcus sp. BV3L6, or Lachnospiraceae bacterium ND2006.

60. The nucleic acid of claim 58 or 59, wherein the Cpf1 polypeptide comprises one or more mutations in a RuvC-like endonuclease domain.

61. The nucleic acid of claim 60, wherein the mutant Cpf1 polypeptide sequence is from Francisella novicida and comprises a mutation in the RuvC-like domain.

62. The nucleic acid of claim 61, wherein the mutation is a D917A, E1006A, or D1225A mutation.

63. The nucleic acid of claim 32, wherein the CRISPR-Cas endonuclease is a Cas9 fusion polypeptide or a Cpf1 fusion polypeptide.

64. The nucleic acid of claim 63, wherein the fusion polypeptide comprises one or more functional domains.

65. The nucleic acid of claim 64, wherein the one or more functional domains is selected from the group consisting of: a histone methylase or demethylase domains, a histone acetylase or deacetylase domains, a SUMOylation domain, an ubiquitylation or deubiquitylation domain, a DNA methylase or DNA demethylase domain, and a nuclease domain.

66. The nucleic acid of claim 65, wherein the nuclease domain is a FOK I nuclease domain.

67. The nucleic acid of claim 65, wherein the nuclease domain is a TREX2 nuclease domain.

68. The nucleic acid of any of the preceding claims, wherein the switch polypeptide comprises a TREX2 domain or is a polypeptide comprising a self-cleaving viral peptide and TREX2.

69. The nucleic acid of any one of preceding claims, wherein the one or more guide RNAs are single strand guide RNAS (sgRNAs).

70. The nucleic acid of any one of preceding claims, wherein the one or more guide RNAs are crRNAs.

71. The nucleic acid of any one of the preceding claims, wherein the polynucleotide encoding the CRISPR-Cas endonuclease further encodes an inhibitory RNA and a binding site for the inhibitory RNA.

72. The nucleic acid of claim 71, wherein the inhibitory RNA is a miRNA or a mishRNA.

73. The nucleic acid of any one of the preceding claims, wherein the polynucleotide encoding the CRISPR-Cas endonuclease further comprises an intron, wherein the intron is spliced in mammalian cells but not in non-mammalian cells.

74. The nucleic acid of claim 73, wherein the intron is an artificial intron.

75. The nucleic acid of claim 73, wherein the intron is a human growth hormone intron.

76. The nucleic acid of claim 73, wherein the intron is an SV40 large T-antigen intron.

77. The nucleic acid of claim 73, wherein the intron is an intron isolated from a mammalian gene.

78. The nucleic acid of any one of the preceding claims, wherein one or more guide RNAs are design to alter at least one site in a genome.

79. The nucleic acid of claim 78, wherein at least one site in the genome is in a gene associated with the signaling of pain.

80. The nucleic acid of claim 78 or claim 79, wherein the at least one site in the genome is in a gene encoding a voltage gated sodium or potassium channel.

81. The nucleic acid of claim 80, wherein the voltage gated sodium channel is selected from the group consisting of: Naᵥ1.1, Naᵥ1.2, Naᵥ1.3, Naᵥ1.5, Naᵥ1.6, Naᵥ1.7, Naᵥ1.8, and Naᵥ1.9.

82. The nucleic acid of any one of claims 78-81, wherein the sequence of the one or more guide RNAs is selected from the group consisting of SEQ ID NOs: 1-55.

83. The nucleic acid of claim 78, wherein the at least one site in the genome is a gene associated with a human channelopathy.

84. The nucleic acid of claim 78, wherein the at least one site in the genome is in a gene set forth in Table 2 or Table 3.

85. The nucleic acid of any one of the preceding claims, further comprising a polynucleotide encoding a template for altering at least one site in a genome.

86. The nucleic acid of claim 85, wherein the template comprises a regulatable transcriptional regulatory element.

87. The nucleic acid of claim 86, wherein the transcriptional regulatory element is targeted for insertion upstream of a transcription start site in a gene of the cell.

88. The nucleic acid of claim 86 or claim 87, wherein the transcriptional regulatory element is activated in the presence of an exogenous ligand or small molecule.

89. The nucleic acid of claim 86 or claim 87, wherein the transcriptional regulatory element is activated in the absence of an exogenous ligand or small molecule.

90. The nucleic acid of claim 86 or claim 87, wherein the transcriptional regulatory element is repressed in the presence of an exogenous ligand or small molecule.

91. The nucleic acid of claim 86 or claim 87, wherein the transcriptional regulatory element is repressed in the absence of an exogenous ligand or small molecule.

92. The nucleic acid of claim 86 or claim 87, wherein the transcriptional regulatory element is inserted upstream of a gene associated with the signaling of pain.

93. The nucleic acid of claim 86 or claim 87, wherein the transcriptional regulatory element is inserted upstream of a gene encoding a voltage gated sodium or potassium channel.

94. The nucleic acid of claim 93, wherein the voltage gated sodium channel is selected from the group consisting of: Naᵥ1.1, Naᵥ1.2, Naᵥ1.3, Naᵥ1.5, Naᵥ1.6, Naᵥ1.7, Naᵥ1.8, and Naᵥ1.9.

95. The nucleic acid of claim 86 or claim 87, wherein the transcriptional regulatory element is inserted upstream of a gene associated with a human channelopathy.

96. The nucleic acid of claim 86 or claim 87, wherein the transcriptional regulatory element is inserted upstream of a gene set forth in Table 2 or Table 3.

97. The nucleic acid of any one of the preceding claims, further comprising an epitope tag.

98. The nucleic acid of claim 97, wherein the epitope tag is selected from the group consisting of: maltose binding protein (“MBP”), glutathione S transferase (GST), HIS6, MYC, FLAG, V5, VSV-G, and HA.

99. The nucleic acid of any one of the preceding claims, further comprising one or more poly(A) sequences.

100. The nucleic acid of claim 99, wherein the one or more poly(A) sequences are selected from the group consisting of: an artifical poly(A) sequence, an SV40 poly(A) sequence, a bovine growth hormone poly(A) sequence (bGHpA), and a rabbit β-globin poly(A) sequence (rβgpA).

101. A vector comprising the nucleic acid of any one of claims 1-100 or as shown in any one of the figures or embodiments disclosed herein.

102. A viral vector comprising the nucleic acid of any one of claims 1-100.

103. An adenoviral vector comprising the nucleic acid of any one of claims 1-100.

104. A lentiviral vector comprising the nucleic acid of any one of claims 1-100.

105. The lentiviral vector of claim 104, wherein the lentivirus is selected from the group consisting of: human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

106. The lentiviral vector of claim 104 or claim 105, wherein the vector comprises a chimeric 5′ LTR.

107. The lentiviral vector of any one of claims 99-101, wherein the vector comprises a 3′ self-inactivating (SIN) LTR.

108. The lentiviral vector of any one of claims 104-107, wherein the vector comprises a cPPT/FLAP sequence.

109. The lentiviral vector of any one of claims 104-108, wherein the vector comprises a woodchuck post-transcriptional regulatory element (WPRE).

110. An adenoviral-associated virus (AAV) vector comprising the nucleic acid of any one of claims 1-100.

111. The AAV vector of claim 110, wherein the AAV vector comprises one or more AAV2 inverted terminal repeats (ITRs).

112. The AAV vector of claim 110 or claim 111, wherein the AAV vector comprises a serotype selected from the group consisting of: AAV1, AAV1(Y705+731F+T492V), AAV2(Y444+500+730F+T491V), AAV3(Y705+731F), AAV5, AAV5(Y436+693+719F), AAV6, AAV6 (VP3 variant Y705F/Y731F/T492V), AAV-7m8, AAV8, AAV8(Y733F), AAV9, AAV9 (VP3 variant Y731F), AAV10(Y733F), and AAV-ShH10.

113. The AAV vector of any one of claims 110-112, wherein the AAV vector comprises a serotype selected from the group consisting of: AAV1, AAV5, AAV6, AAV6 (Y705F/Y731F/T492V), AAV8, AAV9, and AAV9 (Y731F).

114. The AAV vector of any one of claims 110-112, wherein the AAV vector comprises a serotype selected from the group consisting of: AAV6, AAV6 (Y705F/Y731F/T492V), AAV9, and AAV9 (Y731F).

115. The AAV vector of any one of claims 110-112, wherein the AAV vector comprises an AAV6 or AAV6 (Y705F/Y731F/T492V) serotype.

116. The AAV vector of any one of claims 110-115, wherein the AAV vector is a self-complementary AAV (scAAV) vector.

117. A composition comprising the nucleic acid of any one of claims 1-100 and optionally, one or more exosomes, nanoparticles, or biolistics.

118. A composition comprising the vector of any one of claims 102-116.

119. A method of managing, preventing, or treating pain in a subject, comprising administering to the subject the composition of claim 117 or 118.

120. A method of providing analgesia to a subject having pain, comprising administering to the subject the composition of claim 117 or 118.

121. The method of claim 119 or claim 120, wherein the pain is acute pain or chronic pain.

122. The method of any one of claims 119-121, wherein the pain is chronic pain.

123. The method of any one of claims 119-121, wherein the pain is acute pain, chronic pain, neuropathic pain, nociceptive pain, allodynia, inflammatory pain, inflammatory hyperalgesia, neuropathies, neuralgia, diabetic neuropathy, human immunodeficiency virus-related neuropathy, nerve injury, rheumatoid arthritic pain, osteoarthritic pain, burns, back pain, eye pain, visceral pain, cancer pain (e.g., bone cancer pain), dental pain, headache, migraine, carpal tunnel syndrome, fibromyalgia, neuritis, sciatica, pelvic hypersensitivity, pelvic pain, post herpetic neuralgia, post-operative pain, post stroke pain, or menstrual pain.

124. The method of any one of claims 119-121, wherein the pain is nociceptive pain.

125. The method of any one of claims 119-121, wherein the pain is nociceptive pain is selected from the group consisting of central nervous system trauma, strains/sprains, burns, myocardial infarction and acute pancreatitis, post-operative pain (pain following any type of surgical procedure), posttraumatic pain, renal colic, cancer pain and back pain.

126. The method of any one of claims 119-121, wherein the pain is neuropathic pain.

127. The method of claim 126, wherein the etiology of the neuropathic pain is selected from the group consisting of: peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy, and vitamin deficiency.

128. The method of claim 126, wherein the neuropathic pain is related to a pain disorder selected from the group consisting of: arthritis, allodynia, a typical trigeminal neuralgia, trigeminal neuralgia, somatoform disorder, hypoesthesis, hypealgesia, neuralgia, neuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis or pain associated with cancer.

129. The method of any one of claims 119-121, wherein the pain is inflammatory pain.

130. The method of any one of claims 119-121, wherein the pain is associated with musculoskeletal disorders, myalgia, fibromyalgia, spondylitis, sero-negative (non-rheumatoid) arthropathies, non-articular rheumatism, dystrophinopathy, glycogenolysis, polymyositis and pyomyositis; heart and vascular pain, pain caused by angina, myocardical infarction, mitral stenosis, pericarditis, Raynaud's phenomenon, scleredoma and skeletal muscle ischemia; head pain, migraine, cluster headache, tension-type headache mixed headache and headache associated with vascular disorders; orofacial pain, dental pain, otic pain, burning mouth syndrome, and temporomandibular myofascial pain.

131. A method of treating, preventing, ameliorating, or managing a channelopathy in a subject comprising administering the nucleic acid of any one of claims 1-100, the vector of any one of claims 102-116, or the composition of claim 117 or claim 118 to one or more neuronal cells of the subject.

132. The method of claim 131, wherein the channelopathy is associated with a mutation in a voltage gated sodium or potassium channel.

133. The method of claim 132, wherein the voltage gated sodium channel is selected from the group consisting of: Naᵥ1.1, Naᵥ1.2, Naᵥ1.3, Naᵥ1.4, Naᵥ1.5, Naᵥ1.6, Naᵥ1.7, Naᵥ1.8, and Naᵥ1.9.

134. The method of any one of claims 131-133, wherein the channelopathy is selected from the group consisting of: Channelopathy-associated Insensitivity to Pain (CIP), Primary Erythermalgia (PE), Fibromyalgia, Paroxysmal Extreme Pain Disorder (PEPD), Febrile Epilepsy, Generalized Epilepsy with Febrile Seizures, Dravet syndrome, West syndrome, Doose syndrome, Intractable Childhood Epilepsy with Generalized Tonic-Clonic seizures (ICEGTC), Panayiotopoulos syndrome, Familial Hemiplegic Migraine (FHM), Familial Autism, Rasmussen's Encephalitis, Lennox-Gastaut syndrome, Epilepsy, Pain, Hyperkalemic Periodic Paralysis, Paramyotonia Congenita, Potassium-Aggravated Myotonia, Long QT Syndrome, Brugada Syndrome, Idiopathic Ventricular Fibrillation, Irritable Bowel Syndrome, and Neuropsychiatric Disorders.

135. A method of treating, preventing, ameliorating, or managing a disease or condition set forth in Table 2 or Table 3, comprising administering the nucleic acid of any one of claims 1-100, the vector of any one of claims 102-116, or the composition of claim 117 or claim 118 to one or more neuronal cells of the subject.

136. The method of any one of claims 119-135, wherein the nucleic acid of any one of claims 1-100, the vector of any one of claims 102-116, or the composition of claim 117 or claim 118 is intrathecally administered to a subject.

137. The method of any one of claims 119-135, wherein the nucleic acid of any one of claims 1-100, the vector of any one of claims 102-116, or the composition of claim 117 or claim 118 is intraganglionicly administered to a subject.

138. The method of any one of claims 119-135, wherein the nucleic acid of any one of claims 1-100, the vector of any one of claims 102-116, or the composition of claim 117 or claim 118 is intraneurally administered to a subject.

139. The method of any one of claims 119-135, wherein the nucleic acid of any one of claims 1-100, the vector of any one of claims 102-116, or the composition of claim 117 or claim 118 is intramuscularly administered to a subject.

140. The method of any one of claims 119-135, wherein the nucleic acid of any one of claims 1-100, the vector of any one of claims 102-116, or the composition of claim 117 or claim 118 is intracranially administered to a subject.

141. The method of any one of claims 119-135, wherein the nucleic acid of any one of claims 1-100 or the composition of claim 117 or claim 118 is administered to a subject by electroporation.


SEQUENCE LISTING (69 sequences)

SEQ ID NO: 1 - DNA, 23 bp, Artificial Sequence
  sgRNA guide sequence
gtagcagccccgcctccccgagg

SEQ ID NO: 2 - DNA, 23 bp, Artificial Sequence
  sgRNA guide sequence
gggcgcagtctgcttgcaggcgg

SEQ ID NO: 3 - DNA, 23 bp, Artificial Sequence
  sgRNA guide sequence
tgggctagcctgggtgccagtgg

SEQ ID NO: 4 - DNA, 23 bp, Artificial Sequence
  sgRNA guide sequence
tgggtgccagtggctgctagcgg

SEQ ID NO: 5 - DNA, 23 bp, Artificial Sequence
  sgRNA guide sequence
gcagccctgagagagcgccgggg

SEQ ID NO: 6 - DNA, 23 bp, Artificial Sequence
  sgRNA guide sequence
gcagaatctggctccaggagagg

SEQ ID NO: 7 - DNA, 23 bp, Artificial Sequence
  sgRNA guide sequence
gacaaagctctgaggtcctgggg

SEQ ID NO: 8 - DNA, 23 bp, Artificial Sequence
  sgRNA guide sequence
gaccaaaaatgtcgagtaagtgg

SEQ ID NO: 9 - DNA, 23 bp, Artificial Sequence
  sgRNA guide sequence
agagagcgccggggaaggagagg

SEQ ID NO: 10 - DNA, 23 bp, Artificial Sequence
  sgRNA guide sequence
gtgaaatggacaaagctctgagg

SEQ ID NO: 11 - DNA, 23 bp, Artificial Sequence
  sgRNA guide sequence
ggctctgacaccatgccgggagg

SEQ ID NO: 12 - DNA, 23 bp, Artificial Sequence
  sgRNA guide sequence
aggcggggctgctacctccacgg

SEQ ID NO: 13 - DNA, 23 bp, Artificial Sequence
  sgRNA guide sequence
ggcggggctgctacctccacggg

SEQ ID NO: 14 - DNA, 23 bp, Artificial Sequence
  sgRNA guide sequence
agactgcgcccctcctgccaggg

SEQ ID NO: 15 - DNA, 23 bp, Artificial Sequence
  sgRNA guide sequence
gaggggcgcagtctgcttgcagg

SEQ ID NO: 16 - DNA, 23 bp, Artificial Sequence
  sgRNA guide sequence
agcgctccagcggcggctgtcgg

SEQ ID NO: 17 - DNA, 23 bp, Artificial Sequence
  sgRNA guide sequence
ggaaagccgacagccgccgctgg

SEQ ID NO: 18 - DNA, 23 bp, Artificial Sequence
  sgRNA guide sequence
gctttccaattccgccagctcgg

SEQ ID NO: 19 - DNA, 23 bp, Artificial Sequence
  sgRNA guide sequence
gctagcagccactggcacccagg

SEQ ID NO: 20 - DNA, 23 bp, Artificial Sequence
  sgRNA guide sequence
tgccagtggctgctagcggcagg

SEQ ID NO: 21 - DNA, 23 bp, Artificial Sequence
  sgRNA guide sequence
cgcctgccgctagcagccactgg

SEQ ID NO: 22 - DNA, 23 bp, Artificial Sequence
  sgRNA guide sequence
gcaggcgtcccctgagcaacagg

SEQ ID NO: 23 - DNA, 23 bp, Artificial Sequence
  sgRNA guide sequence
agggctgcttctttttctctggg

SEQ ID NO: 24 - DNA, 23 bp, Artificial Sequence
  sgRNA guide sequence
agcagccctgagagagcgccggg

SEQ ID NO: 25 - DNA, 23 bp, Artificial Sequence
  sgRNA guide sequence
gattctgcaggtgcactgggtgg

SEQ ID NO: 26 - DNA, 23 bp, Artificial Sequence
  sgRNA guide sequence
gtgcactgggtggggatgatcgg

SEQ ID NO: 27 - DNA, 23 bp, Artificial Sequence
  sgRNA guide sequence
gtggggatgatcggcgggctagg

SEQ ID NO: 28 - DNA, 23 bp, Artificial Sequence
  sgRNA guide sequence
ggacaaagctctgaggtcctggg

SEQ ID NO: 29 - DNA, 23 bp, Artificial Sequence
  sgRNA guide sequence
tggacaaagctctgaggtcctgg

SEQ ID NO: 30 - DNA, 23 bp, Artificial Sequence
  sgRNA guide sequence
agcaatgcgttgttcaatgaggg

SEQ ID NO: 31 - DNA, 23 bp, Artificial Sequence
  sgRNA guide sequence
ggaatgtccccatagatgaaggg

SEQ ID NO: 32 - DNA, 23 bp, Artificial Sequence
  sgRNA guide sequence
aggaatgtccccatagatgaagg

SEQ ID NO: 33 - DNA, 23 bp, Artificial Sequence
  sgRNA guide sequence
tggggacattcctcccggcatgg

SEQ ID NO: 34 - DNA, 23 bp, Artificial Sequence
  sgRNA guide sequence
cggcatggtgtcagagcccctgg

SEQ ID NO: 35 - DNA, 23 bp, Artificial Sequence
  sgRNA guide sequence
gacagccgccgctggagcgctgg

SEQ ID NO: 36 - DNA, 23 bp, Artificial Sequence
  sgRNA guide sequence
gagaggcccgcgccctctcctgg

SEQ ID NO: 37 - DNA, 23 bp, Artificial Sequence
  sgRNA guide sequence
gccagattctgcaggtgcactgg

SEQ ID NO: 38 - DNA, 23 bp, Artificial Sequence
  sgRNA guide sequence
aggggctctgacaccatgccggg

SEQ ID NO: 39 - DNA, 23 bp, Artificial Sequence
  sgRNA guide sequence
cggctgaggctgggctagcctgg

SEQ ID NO: 40 - DNA, 23 bp, Artificial Sequence
  sgRNA guide sequence
agctcctcacataagaggcctgg

SEQ ID NO: 41 - DNA, 23 bp, Artificial Sequence
  sgRNA guide sequence
gtctgcaggaggaaaaagaaagg

SEQ ID NO: 42 - DNA, 23 bp, Artificial Sequence
  sgRNA guide sequence
gtaggggtccaagtcctccaggg

SEQ ID NO: 43 - DNA, 23 bp, Artificial Sequence
  sgRNA guide sequence
agtaggggtccaagtcctccagg

SEQ ID NO: 44 - DNA, 23 bp, Artificial Sequence
  sgRNA guide sequence
gtcagagcccctggaggacttgg

SEQ ID NO: 45 - DNA, 23 bp, Artificial Sequence
  sgRNA guide sequence
ggctgaggctgggctagcctggg

SEQ ID NO: 46 - DNA, 23 bp, Artificial Sequence
  sgRNA guide sequence
cggcgcagctgccctcggggagg

SEQ ID NO: 47 - DNA, 23 bp, Artificial Sequence
  sgRNA guide sequence
cgcagctgccctcggggaggcgg

SEQ ID NO: 48 - DNA, 23 bp, Artificial Sequence
  sgRNA guide sequence
cgggcgcgccctggcaggagggg

SEQ ID NO: 49 - DNA, 23 bp, Artificial Sequence
  sgRNA guide sequence
ggcggtcgccagcgctccagcgg

SEQ ID NO: 50 - DNA, 23 bp, Artificial Sequence
  sgRNA guide sequence
ggtcgccagcgctccagcggcgg

SEQ ID NO: 51 - DNA, 23 bp, Artificial Sequence
  sgRNA guide sequence
ggcgcgggcctctccttccccgg

SEQ ID NO: 52 - DNA, 23 bp, Artificial Sequence
  sgRNA guide sequence
ggagaaagcatatataaagcagg

SEQ ID NO: 53 - DNA, 23 bp, Artificial Sequence
  sgRNA guide sequence
gtgtttaggtacacttttactgg

SEQ ID NO: 54 - DNA, 23 bp, Artificial Sequence
  sgRNA guide sequence
gcagctgccctcggggaggcggg

SEQ ID NO: 55 - DNA, 23 bp, Artificial Sequence
  sgRNA guide sequence
gaagaggaattaaaatatacagg

SEQ ID NO: 56 - DNA, 10 bp, Artificial Sequence
  Consensus Kozak sequence
gccrccatgg

SEQ ID NO: 57 - protein, 5 aa, Artificial Sequence
  Exemplary linker sequence
DGGGS

SEQ ID NO: 58 - protein, 5 aa, Artificial Sequence
  Exemplary linker sequence
TGEKP

SEQ ID NO: 59 - protein, 4 aa, Artificial Sequence
  Exemplary linker sequence
GGRR

SEQ ID NO: 60 - protein, 5 aa, Artificial Sequence
  Exemplary linker sequence
GGGGS

SEQ ID NO: 61 - protein, 14 aa, Artificial Sequence
  Exemplary linker sequence
EGKSSGSGSESKVD

SEQ ID NO: 62 - protein, 18 aa, Artificial Sequence
  Exemplary linker sequence
KESGSVSSEQLAQFRSLD

SEQ ID NO: 63 - protein, 8 aa, Artificial Sequence
  Exemplary linker sequence
GGRRGGGS

SEQ ID NO: 64 - protein, 9 aa, Artificial Sequence
  Exemplary linker sequence
LRQRDGERP

SEQ ID NO: 65 - protein, 12 aa, Artificial Sequence
  Exemplary linker sequence
LRQKDGGGSERP

SEQ ID NO: 66 - protein, 16 aa, Artificial Sequence
  Exemplary linker sequence
LRQKDGGGSGGGSERP

SEQ ID NO: 67 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 68 - protein, 7 aa, Artificial Sequence
  Cleavage sequence by TEV protease
ENLYFQG

SEQ ID NO: 69 - protein, 7 aa, Artificial Sequence
  Cleavage sequence by TEV protease
ENLYFQS